(19) United States
(12) Patent Application Publication
(10) Pub. No.: US 2025/0095826 A1
(43) Pub. Date: Mar. 20, 2025

(54) ENSEMBLED QUERYING OF EXAMPLE IMAGES VIA DEEP LEARNING EMBEDDINGS

(71) Applicant: GE Precision Healthcare LLC, Waukesha, WI (US)

(72) Inventors: Vikram Reddy Melapudi, Bangalore (IN); Hariharan Ravishankar, Bengaluru (IN); Deepa Anand, Bangalore (IN)

(21) Appl. No.: 18/470,734

(22) Filed: Sep. 20, 2023

(51) Int. Cl. G16H 30/40; G06F 3/14; G06T 7/11; G06T 7/70; G06V 10/82

(52) U.S. Cl. CPC G16H 30/40; G06F 3/14; G06T 7/11; G06T 7/70; G06V 10/82

(57) ABSTRACT

Systems or techniques that facilitate ensembled querying of example images via deep learning embeddings are provided. In various embodiments, a system can access a medical image associated with a medical patient. In various aspects, the system can generate an ensembled heat map indicating where in the medical image an anatomical structure is likely to be located, by executing an embedder neural network on the medical image and on a plurality of example medical images associated with other medical patients. In various instances, respective instantiations of the anatomical structure can be flagged in the plurality of example medical images by user-provided clicks.

### TECHNICAL FIELD

The subject disclosure relates generally to deep learning, and more specifically to ensembled querying of example images via deep learning embeddings.

### BACKGROUND

When given a medical image, it can be desired to localize an anatomical structure that is depicted in the medical image. Existing techniques facilitate such localization by training a deep learning neural network in supervised fashion. Unfortunately, such existing techniques rely upon the availability of voluminous annotated training data, which can be excessively time-consuming or difficult to acquire. Furthermore, the deep learning neural networks trained according to such existing techniques exhibit excessively poor generalizability.

Accordingly, systems or techniques that can facilitate anatomical structure localization without the training data acquisition costs and the restricted generalizability of existing techniques can be considered as desirable.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products that facilitate ensembled querying of example images via deep learning embeddings are described.

According to one or more embodiments, a system is provided. The system can comprise a non-transitory computer-readable memory that can store computer-executable components. The system can further comprise a processor that can be operably coupled to the non-transitory computer-readable memory and that can execute the computer-executable components stored in the non-transitory computer-readable memory. In various embodiments, the computer-executable components can comprise an access component that can access a medical image associated with a medical patient. In various aspects, the computer-executable components can comprise a localization component that can generate an ensembled heat map indicating where in the medical image an anatomical structure is likely to be located, by executing an embedder neural network on the medical image and on a plurality of example medical images associated with other medical patients, wherein respective instantiations of the anatomical structure can be flagged in the plurality of example medical images by user-provided clicks.

According to one or more embodiments, a computer-implemented method is provided. In various embodiments, the computer-implemented method can comprise accessing, by a device operatively coupled to a processor, a medical image associated with a medical patient. In various aspects, the computer-implemented method can comprise generating, by the device, an ensembled heat map indicating where in the medical image an anatomical structure is likely to be located, by executing an embedder neural network on the medical image and on a plurality of example medical images associated with other medical patients, wherein respective instantiations of the anatomical structure can be flagged in the plurality of example medical images by user-provided clicks.

According to one or more embodiments, a computer program product for facilitating ensembled querying of example images via deep learning embeddings is provided. In various embodiments, the computer program product can comprise a non-transitory computer-readable memory having program instructions embodied therewith. In various aspects, the program instructions can be executable by a processor to cause the processor to access an image. In various instances, the program instructions can be further executable to cause the processor to localize an object of interest depicted in the image, by executing an embedder neural network on the image and on a plurality of example images, wherein respective instantiations of the object of interest can be flagged in the plurality of example images by user-provided clicks.

### DESCRIPTION OF THE DRAWINGS

The patent or application file contains at least one drawing executed in color. Copies of this patent or patent application publication with color drawing(s) will be provided by the Office upon request and payment of the necessary fec.

FIG. 1 illustrates a block diagram of an example, non-limiting system that facilitates ensembled querying of example images via deep learning embeddings in accordance with one or more embodiments described herein.

FIG. 2 illustrates a block diagram of an example, non-limiting system including a plurality of example medical images and corresponding user-provided clicks that facilitates ensembled querying of example images via deep learning embeddings in accordance with one or more embodiments described herein.

FIG. 3 illustrates an example, non-limiting block diagram showing a plurality of example medical images and corresponding user-provided clicks in accordance with one or more embodiments described herein.

FIG. 4 illustrates a block diagram of an example, non-limiting system including an embedder neural network and an ensembled heat map that facilitates ensembled querying of example images via deep learning embeddings in accordance with one or more embodiments described herein.

FIGS. 5-9 illustrate example, non-limiting block diagrams showing how an embedder neural network can be implemented to generate an ensembled heat map in accordance with one or more embodiments described herein.

FIG. 10 illustrates a block diagram of an example, non-limiting system including a training component that facilitates ensembled querying of example images via deep learning embeddings in accordance with one or more embodiments described herein.

FIG. 11 illustrates an example, non-limiting block diagram showing how an embedder neural network can be trained in accordance with one or more embodiments described herein.

FIGS. 12-17 illustrate example, non-limiting experimental results in accordance with one or more embodiments described herein.

FIG. 18 illustrates a flow diagram of an example, non-limiting computer-implemented method that facilitates ensembled querying of example images via deep learning embeddings in accordance with one or more embodiments described herein.

FIG. 19 illustrates a block diagram of an example, non-limiting operating environment in which one or more embodiments described herein can be facilitated.

FIG. 20 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

When given a medical image, it can be desired to localize an anatomical structure (e.g., an organ, a tissue, a body part, or a portion thereof) that is visually depicted or illustrated in the medical image. In other words, it can be desired to determine which specific pixels (or voxels) of the medical image belong to or otherwise make up the anatomical structure.

Existing techniques facilitate such localization by training a deep learning neural network in supervised fashion. In particular, such existing techniques involve acquiring or curating an annotated training dataset, where the annotated training dataset comprises various training medical images and various ground-truth annotations that respectively correspond to the training medical images. A training medical image can depict an anatomical structure of interest, and a ground-truth annotation corresponding to that training medical image can indicate a correct or accurate intra-image location of that anatomical structure of interest within that training medical image. For example, the ground-truth annotation can be a segmentation mask or a bounding box indicating where within that training medical image the anatomical structure of interest is known or deemed to be located.

Unfortunately, such existing techniques suffer from various disadvantages. In particular, in order for the deep learning neural network to achieve acceptable localization accuracy or precision, such existing techniques require that the annotated training dataset be sufficiently voluminous. That is, such existing techniques require that the deep learning neural network be trained on very many training medical images and very many ground-truth annotations. Given the sensitive, private, and highly regulated nature of medical information, curation or acquisition of such voluminous training data can consume excessive amounts of time, effort, or other expense on the part of technicians that oversee the deep learning neural network.

Furthermore, even if such training data curation or acquisition difficulties are surmounted, the deep learning neural network trained according to such existing techniques often exhibits excessively poor generalizability. More specifically, by being trained on the annotated training dataset, the deep learning neural network learns how to accurately or reliably localize only the types or classes of anatomical structures that are included or otherwise represented in the annotated training dataset. In other words, the deep learning neural network cannot accurately or reliably localize types or classes of anatomical structures that were not included or otherwise represented in the annotated training dataset. For example, suppose that the ground-truth annotations of the annotated training dataset indicate only the correct or accurate intra-image locations of brain tumors that are illustrated in respective training medical images. In such case, the deep learning neural network would, after training, be able to accurately or reliably localize only brain tumors and would not be able to accurately or reliably localize any anatomical structures that are not brain tumors (e.g., would not be able to accurately or reliably localize lung lesions, occluded blood vessels, or bone fractures). That is, the deep learning neural network would not be able to effectively generalize beyond brain tumor localization, without undergoing extensive retraining or fine-tuning.

Accordingly, systems or techniques that can facilitate anatomical structure localization without the training data acquisition difficulties and without the restricted generalizability of existing techniques can be considered as desirable.

Various embodiments described herein can address one or more of these technical problems. One or more embodiments described herein can include systems, computer-implemented methods, apparatus, or computer program products that can facilitate ensembled querying of example images via deep learning embeddings. In other words, the inventors of various embodiments described herein devised various techniques that utilize embeddings (e.g., latent vector representations) to facilitate anatomical structure localization. In particular, when given a medical image, any suitable anatomical structure of interest can be localized within the given medical image, by executing an embedder neural network on the given medical image and on two or more example medical images, where the two or more example medical images can illustrate respective instantiations of the anatomical structure of interest, and where such respective instantiations can be called-out or otherwise marked by user-provided clicks (e.g., clicks inputted by a technician via a computer mouse or touchscreen). In various aspects, a user-provided click can be considered as indicating, identifying, or otherwise flagging, within a respective example medical image, a region or patch of pixels (or voxels) in which an instantiation of the anatomical structure of interest is known or deemed to be located. In various instances, the embedder neural network can generate an embedding for the indicated, identified, or flagged region or patch of the respective example medical image, and the embedder neural network can further generate embeddings for all regions or patches of the given medical image. In various cases, the anatomical structure of interest can be localized within the given medical image, by comparing the embedding of that indicated, identified, or flagged region or patch with each of the embeddings of the regions or patches of the given medical image. In various aspects, such comparison can be considered as a search or query for regions or patches of the given medical image whose embeddings (and, thus, whose visual contents) are similar to that of the indicated, identified, or flagged region or patch of the respective example medical image. In various instances, such embedding comparison can yield a heat map that indicates which regions or patches of the given medical image are more similar or less similar to the indicated, identified, or flagged region or patch of the respective example medical image. In various cases, such embedding comparison can be performed for each of the two or more example medical images, thereby yielding two or more heat maps (e.g., one heat map per example medical image), and such two or more heat maps can be aggregated together into an ensembled heat map that reliably shows which regions or patches of the given medical image are more or less likely to depict the anatomical structure of interest.

Note that various embodiments described herein can be considered as addressing or ameliorating various disadvantages of existing techniques. Indeed, as mentioned above, a deep learning neural network trained according to existing techniques learns in supervised fashion based on a voluminous amount of annotated training data (e.g., on thousands of annotated training medical images). In stark contrast, the embedder neural network described herein can instead be trained in unsupervised fashion (e.g., trained in an encoder-decoder deep learning pipeline; trained in a self-distillation-no-labels fashion). Such unsupervised training can be facilitated in the absence of ground-truth annotations, which can significantly reduce training data curation or acquisition effort. Furthermore, as described herein, the embedder neural network can be configured to operate on individual regions or patches of medical images rather than on entireties of medical images. Thus, such unsupervised training can be facilitated using only regions or patches of training medical images, rather than using full or entire training medical images. Again, this can help to reduce training data curation or acquisition effort. Further still, as described herein, such unsupervised training need not even involve medical images at all. Indeed, regions or patches of any suitable images (even non-medical images) can be used to train the embedder neural network to produce embeddings. Once again, this can help to reduce training data curation or acquisition effort.

Although the embedder neural network can be trained in unsupervised fashion, various embodiments described herein nevertheless involve two or more example medical images having respective user-provided clicks that mark or flag respective instantiations of an anatomical structure of interest. However, such two or more example medical images are not utilized for training, and curation or acquisition of such two or more example medical images can be considered as easy or otherwise non-burdensome compared to the data curation or acquisition of existing techniques. Indeed, the two or more example medical images can be many orders of magnitude fewer in cardinality than the training data required by existing techniques (e.g., existing techniques can require thousands of annotated training medical images, whereas various embodiments described herein can involve merely two, three, or four example medical images). Additionally, unlike existing techniques, the two or more example medical images need not correspond to full segmentation masks or full bounding boxes that indicate intra-image locations of anatomical structures of interest. Instead, each example medical image can have a respective user-provided click that is known or deemed to be on an anatomical structure of interest, the example medical image can be broken up into regular and disjoint regions or patches of pixels (or voxels), and whichever region or patch contains the user-provided click can be considered as depicting the anatomical structure of interest. It can be much easier and much less time-consuming for a technician to supply a user-provided click than to supply a full segmentation mask or bounding box.

Moreover, various embodiments described herein can be considered as facilitating universally generalizable anatomical structure localization. Indeed, as mentioned above, a deep learning neural network trained according to existing techniques learns how to localize only the specific types or classes of anatomical structures that are included or represented in its annotated training dataset. In stark contrast, various embodiments described herein can be implemented to localize whatever anatomical structures are flagged or called-out in the two or more example medical images, no matter their types or classes. After all, the embedder neural network, as described herein, can be trained to generate embeddings for inputted regions or patches of pixels (or voxels), regardless of the visual contents of those inputted regions or patches. So, the embedder neural network can produce embeddings for the regions or patches of the two or more example medical images that are indicated, identified, or flagged by the user-provided clicks, no matter what type or class of anatomical structure is depicted in such regions or patches, and such embeddings can be leveraged to localize that same anatomical structure (whatever it may be) within the given medical image. On the other hand, deep learning neural networks trained according to existing techniques cannot accurately or reliably generalize any anatomical structure that was not explicitly included in its annotated training dataset, absent significant retraining or fine-tuning.

Additionally, as described herein, implementation of two or more example medical images can help to improve reliability of anatomical structure localization. After all, it can be possible for any single example medical image to have visual artifacts or to otherwise be visually unusual (e.g., to be unexpectedly distorted, to have unexpected brightness or contrast, to have unexpected blurring), and such visual artifacts or visual unusualness can yield incorrect or inaccurate localization results. To overcome this issue, multiple example medical images can be utilized, so that the likelihood of any single errant example medical image derailing the localization results is reduced. In other words, an ensemble of example medical images can be queried with deep learning embeddings as described herein, so as to facilitate robust anatomical structure localization.

Various embodiments described herein can be considered as a computerized tool (e.g., any suitable combination of computer-executable hardware or computer-executable software) that can facilitate ensembled querying of example images via deep learning embeddings. In various aspects, such computerized tool can comprise an access component, an example component, a localization component, or a display component.

In various embodiments, there can be a particular medical image. In various aspects, the particular medical image can exhibit any suitable format, size, or dimensionality (e.g., the particular medical image can be a two-dimensional pixel array, or the particular medical image can be a three-dimensional voxel array). In various instances, the particular medical image can be generated or captured by any suitable medical imaging modality or equipment (e.g., generated or captured by a computed tomography (CT) scanner, by a magnetic resonance imaging (MRI) scanner, by an X-ray scanner, by an ultrasound scanner, or by a positron emission tomography (PET) scanner). In various cases, the particular medical image can visually depict any suitable anatomical structure of interest of any suitable medical patient.

It can be desired to localize the anatomical structure of interest within the particular medical image, without experiencing the training data acquisition and generalizability difficulties that plague existing techniques. The computerized tool described herein can facilitate such localization.

In various embodiments, the access component of the computerized tool can electronically receive or otherwise electronically access the particular medical image. In some aspects, the access component can electronically retrieve the particular medical image from any suitable centralized or decentralized data structures (e.g., graph data structures, relational data structures, hybrid data structures), whether remote from or local to the access component. In any case, the access component can electronically obtain or access the particular medical image, such that other components of the computerized tool can electronically interact with (e.g., read, write, edit, copy, manipulate) the particular medical image.

In various embodiments, the example component of the computerized tool can electronically access, from any suitable electronic source, a plurality of example medical images. In various aspects, each example medical image can be any suitable medical image that is different or distinct from the particular medical image but that nevertheless depicts an instantiation or version of the anatomical structure of interest. More specifically, each example medical image can be considered as illustrating the anatomical structure of interest of some respective medical patient, where that respective medical patient is different from or otherwise not related to the medical patient whose anatomical structure of interest is illustrated in the particular medical image.

As a non-limiting example, suppose that the anatomical structure of interest is a carotid artery, and suppose that the two or more example medical images comprise three example medical images in total: a first example medical image; a second example medical image; and a third example medical image. In such case, the particular medical image can be considered as depicting the carotid artery of a medical patient A, the first example medical image can be considered as depicting the carotid artery of a medical patient B, the second example medical image can be considered as depicting the carotid artery of a medical patient C, and the third example medical image can be considered as depicting the carotid artery of a medical patient D, where the medical patients A, B, C, and D are different or otherwise not identical. Because the medical patients A, B, C, and D are different or otherwise not identical, it can be the case that the anatomical structure of interest exhibits slightly (or, in some cases, significantly) different visual characteristics (e.g., size, shape, color) across the particular medical image and the first, second, and third example medical images. Furthermore, because the medical patients A, B, C, and D are different or otherwise not identical, it can also be the case that the particular medical image and the first, second, and third example medical images are not fully or completely aligned or registered with each other.

In any case, each of the two or more example medical images can be annotated or otherwise associated with a respective user-provided click indicating, marking, or otherwise flagging where the anatomical structure of interest is located within that example medical image. In various aspects, a medical professional or other expert can supply the user-provided click for any example medical image, via any suitable human-computer interface device. As a non-limiting example, an example medical image can be visually displayed on a computer screen, and the medical professional or other expert can use a computer mouse (or touchscreen functionality if the computer screen is so equipped) to click on whatever intra-image location that the medical professional or other expert knows or deems belongs to the anatomical structure of interest.

Note that a user-provided click differs from a full-blown segmentation mask or a full-blown bounding box (e.g., it can be much less burdensome and time-consuming for the medical professional or other expert to supply a single click for each example medical image, as compared to instead crafting an entire ground-truth segmentation mask or an entire ground-truth bounding box for each example medical image).

Furthermore, note that the medical professional or other expert that supplied the user-provided clicks might be unavailable at whatever time that the particular medical image is encountered or accessed. Despite such absence of the medical professional or other expert, the anatomical structure can nevertheless be localized within the particular medical image, by leveraging the two or more example medical images, as described herein.

In various embodiments, the localization component of the computerized tool can electronically localize the anatomical structure of interest within the particular medical image, based on the two or more example medical images.

More specifically, in various aspects, the localization component can store, maintain, control, or otherwise access an embedder neural network. In various aspects, the embedder neural network can exhibit any suitable internal architecture. For example, the embedder neural network can include any suitable numbers of any suitable types of layers (e.g., input layer, one or more hidden layers, output layer, any of which can be convolutional layers, dense layers, non-linearity layers, pooling layers, batch normalization layers, or padding layers). As another example, the embedder neural network can include any suitable numbers of neurons in various layers (e.g., different layers can have the same or different numbers of neurons as each other). As yet another example, the embedder neural network can include any suitable activation functions (e.g., softmax, sigmoid, hyperbolic tangent, rectified linear unit) in various neurons (e.g., different neurons can have the same or different activation functions as each other). As still another example, the embedder neural network can include any suitable interneuron connections or interlayer connections (e.g., forward connections, skip connections, recurrent connections).

Regardless of its internal architecture, the embedder neural network can be configured to compress regions or patches of pixels (or voxels) into embeddings. Accordingly, in various aspects, the localization component can execute the embedder neural network in region-wise or patch-wise fashion on the particular medical image and on each of the two or more example medical images, and the localization component can localize the anatomical structure of interest within the particular medical image based on embeddings produced by the embedder neural network.

More specifically, the particular medical image can be considered as being composed or otherwise made up of a set of pixel (or voxel) patches. In various aspects, the set of pixel (or voxel) patches can be regularly shaped (e.g., each patch can be a rectilinear array of pixels or voxels) and disjoint (e.g., non-overlapping) with each other. Equivalently, the set of pixel (or voxel) patches can be considered as tiles that, when placed together, collectively form the particular medical image.

In various instances, the localization component can execute the embedder neural network on each of the set of pixel (or voxel) patches, thereby yielding a set of patch embeddings. In particular, for any given pixel (or voxel) patch, the localization component can feed that given pixel (or voxel) patch to an input layer of the embedder neural network, that given pixel (or voxel) patch can complete a forward pass through one or more hidden layers of the embedder neural network, and an output layer of the embedder neural network can compute an embedding for that given pixel (or voxel) patch, based on activations from the one or more hidden layers of the embedder neural network. In various cases, the embedding can be considered as a latent vector representation of that given pixel (or voxel) patch. Accordingly, the embedding can be any suitable electronic data whose format, size, or dimensionality is smaller (e.g., many orders of magnitude smaller) than that of the given pixel (or voxel) patch but that nevertheless indicates or conveys (e.g., albeit in a hidden fashion) visual content of the given pixel (or voxel) patch. For example, the given pixel (or voxel) patch can comprise dozens of pixels (or voxels), and the embedding can be a vector having merely a small handful of elements; however, that small handful of elements can collectively represent, indicate, or otherwise capture at least some of whatever substantive visual content is illustrated by those dozens of pixels (or voxels). Thus, the embedder neural network can, in various instances, be considered as having compressed the given pixel (or voxel) patch into the embedding.

In any case, executing the embedder neural network on each of the set of pixel (or voxel) patches of the particular medical image can yield a set of patch embeddings that respectively correspond to the set of pixel (or voxel) patches.

Now, just as the particular medical image can be considered as being composed or made up of a set of pixel (or voxel) patches, each of the two or more example medical images can likewise be considered as being composed or made up of a respective set of pixel (or voxel) patches. In various aspects, for any given example medical image, one of the set of pixel (or voxel) patches that compose or make up that given example medical image can be considered as being flagged by the user-provided click of that given example medical image. In particular, whichever pixel (or voxel) patch that contains the user-provided click (e.g., inside of which the user-provided click is located) can be considered as being flagged. Accordingly, since each of the two or more example medical images can have a respective user-provided click, there can be two or more flagged pixel (or voxel) patches (e.g., one flagged patch per example medical image).

In various aspects, the localization component can execute the embedder neural network on each of the two or more flagged pixel (or voxel) patches, thereby yielding two or more flagged patch embeddings. In particular, for any given flagged pixel (or voxel) patch, the localization component can feed that given flagged pixel (or voxel) patch to the input layer of the embedder neural network, that given flagged pixel (or voxel) patch can complete a forward pass through the one or more hidden layers of the embedder neural network, and the output layer of the embedder neural network can compute an embedding for that given flagged pixel (or voxel) patch, based on activations from the one or more hidden layers of the embedder neural network.

In any case, executing the embedder neural network on each of the two or more flagged pixel (or voxel) patches can yield a two or more flagged patch embeddings that respectively correspond to the two or more flagged pixel (or voxel) patches.

In various aspects, for any given example medical image, the localization component can generate a heat map by comparing the flagged patch embedding of that given example medical image to each of the set of patch embeddings of the particular medical image. In various instances, the heat map can be considered as comprising similarity scores (e.g., cosine similarity values) that are computed between the flagged patch embedding of the given example medical image and each of the set of patch embeddings of the particular medical image. In other words, the heat map can be considered as indicating how similar or how dissimilar each pixel (or voxel) patch of the particular medical image is to the flagged pixel (or voxel) patch of the given example medical image. Because the flagged pixel (or voxel) patch of the given example medical image can be known or deemed to depict an instantiation of the anatomical structure of interest, the heat map can thus be considered as indicating which specific pixel (or voxel) patches of the particular medical image are more likely or less likely to depict the anatomical structure of interest. In this way, the localization component can generate a respective heat map for each of the two or more example medical images, thereby yielding two or more heat maps.

In various instances, the localization component can aggregate (e.g., via summing or averaging) the two or more heat maps together, thereby yielding an ensembled heat map. In various cases, the ensembled heat map can be considered as representing which specific pixel (or voxel) patches of the particular medical image are likely or not likely to depict the anatomical structure of interest (e.g., to depict whatever visual content is flagged or called-out by the user-provided clicks of the two or more example medical images). That is, the ensembled heat map can be considered as a localizing the anatomical structure of interest within the particular medical image.

In this way, the computerized tool can, in various embodiments, facilitate localization of whatever anatomical structure of interest is called-out or flagged in the two or more example medical images by the user-provided clicks. In other words, the computerized tool can facilitate such localization regardless of the type or class of the anatomical structure of interest. In still other words, the computerized tool can be considered as facilitating universally generalizable anatomical structure localization (also referred to as open vocabulary localization).

In various embodiments, the display component of the computerized tool can electronically render, on any suitable electronic display, the ensembled heat map. Accordingly, the ensembled heat map can be viewable by a user, operator, or technician associated with the computerized tool. In various aspects, the ensembled heat map can be considered as useful for diagnostic or prognostic purposes with respect to the medical patient whose anatomy is illustrated by the particular medical image.

In order for the embeddings described herein to be accurate, the embedder neural network can first undergo training. Accordingly, the computerized tool can comprise a training component, and the training component can train the embedder neural network in any suitable fashion. As a non-limiting example, the training component can train the embedder neural network in unsupervised fashion using an encoder-decoder deep learning framework. As another non-limiting example, the training component can train the embedder neural network via a self-distillation-with-no-labels (DINO) technique.

Various embodiments described herein can be employed to use hardware or software to solve problems that are highly technical in nature (e.g., to facilitate ensembled querying of example images via deep learning embeddings), that are not abstract and that cannot be performed as a set of mental acts by a human. Further, some of the processes performed can be performed by a specialized computer (e.g., a deep learning neural network having internal parameters such as convolutional kernels) for carrying out defined acts related to deep learning.

For example, such defined acts can include: accessing, by a device operatively coupled to a processor, a medical image associated with a medical patient; and generating, by the device, an ensembled heat map indicating where in the medical image an anatomical structure is likely to be located, by executing an embedder neural network on the medical image and on a plurality of example medical images associated with other medical patients, wherein respective instantiations of the anatomical structure can be flagged in the plurality of example medical images by user-provided clicks. In various cases, the embedder neural network can be executed on the medical image and on the plurality of example medical images in patch-wise fashion.

Such defined acts are not performed manually by humans. Indeed, neither the human mind nor a human with pen and paper can electronically access a first medical image, electronically access multiple second medical images in which instantiations of some anatomical structure are flagged by user-provided clicks, and electronically localize the anatomical structure in the first medical image by executing an embedder neural network in patch-wise fashion on both the first medical image and the multiple second medical images. Indeed, a deep learning neural network is an inherently-computerized construct that simply cannot be meaningfully executed or trained in any way by the human mind without computers. Furthermore, deep learning embeddings are latent vector representations of electronic data that also cannot be meaningfully implemented in any way by the human mind with computers. Accordingly, a computerized tool that can localize anatomical structures via patch-wise execution of an embedder neural network on an ensemble of example medical images is likewise inherently-computerized and cannot be implemented in any sensible, practical, or reasonable way without computers.

Moreover, various embodiments described herein can integrate into a practical application various teachings relating to ensembled querying of example images via deep learning embeddings. As explained above, when a deep learning neural network is trained to localize an anatomical structure according to existing techniques, a voluminous annotated training dataset is required (e.g., thousands of training images with corresponding segmentation masks or bounding boxes), which can be highly time-consuming or difficult to obtain. Additionally, the deep learning neural network, when trained according to existing techniques, cannot generalize beyond whatever anatomical structures are explicitly included in the voluminous annotated training dataset, without undergoing extensive retraining. For at least these reasons, existing techniques can be considered as disadvantageous.

Various embodiments described herein can address one or more of these technical problems. Specifically, various embodiments described herein can localize an anatomical structure of interest within a given medical image, by utilizing deep learning embeddings (e.g., latent vectors) to query flagged, called-out, or clicked pixel (or voxel) patches of multiple example medical images that are known to depict an anatomical structure of interest. In particular, the given medical image can be decomposed into a set of patches, and an embedder neural network can be executed on each of such set of patches, thereby yielding a set of first embeddings. Furthermore, each example medical image can have a patch that has been flagged or called-out (e.g., by a user-provided click) as depicting an instantiation or version of the anatomical structure of interest, and the embedder neural network can be executed on that flagged or called-out patch, thereby yielding a second embedding. In various aspects, a heat map corresponding to that example medical image can be generated, by computing a similarity score between the second embedding and each of the set of first embeddings. This can be performed for each of the multiple example medical images, thereby yielding multiple heat maps. In various instances, such multiple heat maps can be aggregated together into a single, ensembled heat map that shows which patches of the given medical image are more or less likely to contain the anatomical structure of interest.

Note that the embedder neural network described herein can be trained in unsupervised fashion (e.g., in an encoder-decoder deep learning pipeline, or in a DINO fashion). Such unsupervised training can involve significantly less training data acquisition or curation as compared to the supervised training of existing techniques. After all, the embedder neural network as described herein can be trained without ground-truth annotations and can be trained using only patches of pixels or voxels rather than full training images. Moreover, the patches of pixels or voxels on which the embedder neural network is trained can come from any suitable types of images, even non-medical images. For at least these reasons, it can take less time or effort to train the embedder neural network than to train a deep learning neural network according to existing techniques.

Although various embodiments described herein do involve acquisition or curation of multiple example medical images, such acquisition or curation can nevertheless be significantly less time-consuming or effort-intensive than the acquisition or curation of training data for existing techniques. After all, various embodiments described herein can function using merely a small handful of example medical images (e.g., five or fewer example medical images), whereas existing techniques require on the order of thousands of training medical images. Furthermore, each example medical image described herein need not correspond to a full-blown segmentation mask or bounding box. Instead, each example medical image described herein can correspond to a respective user-provided click, which can be much easier to obtain than full-blown segmentation masks or bounding boxes.

Additionally, various embodiments described herein can be considered as facilitating universally generalizable anatomical structure localization. After all, the embeddings produced by the embedder neural network can be used to identify patches or regions within a given medical image that have the same visual content as whatever patches or regions are flagged in the example medical images, no matter that visual content. So, if the anatomical structure of interest is flagged in the example medical images, that anatomical structure of interest can be localized within the given medical image, regardless of a type or class of the anatomical structure of interest. In stark contrast, a deep learning neural network trained according to existing techniques can accurately or reliably localize only the types or classes of anatomical structures that are explicitly included in the data on which it is trained.

For at least these reasons, various embodiments described herein certainly constitute concrete and tangible technical improvements in the field of deep learning, and such embodiments therefore clearly qualify as useful and practical applications of computers.

Furthermore, various embodiments described herein can control real-world tangible devices based on the disclosed teachings. For example, various embodiments described herein can electronically execute or train real-world deep learning neural networks and can electronically render, on computer screens, real-world results (e.g., heat maps) computed by such real-world deep learning neural networks.

It should be appreciated that the herein figures and description provide non-limiting examples of various embodiments and are not necessarily drawn to scale.

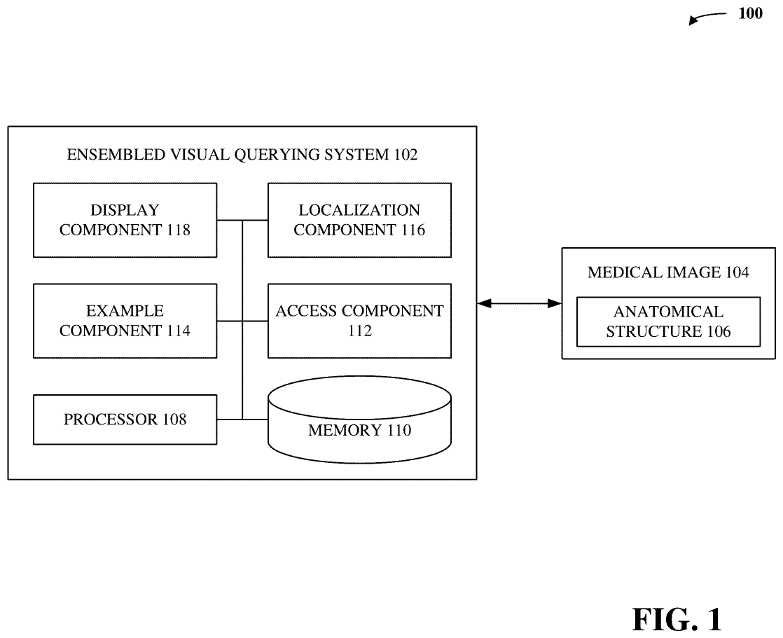

FIG. 1 illustrates a block diagram of an example, non-limiting system 100 that can facilitate ensembled querying of example images via deep learning embeddings in accordance with one or more embodiments described herein. As shown, an ensembled visual querying system 102 can be electronically integrated, via any suitable wired or wireless electronic connections, with a medical image 104.

In various embodiments, the medical image 104 can be any suitable image exhibiting any suitable format, size, or dimensionality. As a non-limiting example, the medical image 104 can be an x-by-y array of pixels, for any suitable positive integers x and y. As another non-limiting example, the medical image 104 can be an x-by-y-by-z array of voxels, for any suitable positive integers x, y, and z. In various aspects, the medical image 104 can be captured or otherwise generated by any suitable medical imaging modality. As a non-limiting example, the medical image 104 can be captured or generated by a CT scanner, in which case the medical image 104 can be considered as a CT scanned image. As another non-limiting example, the medical image 104 can be captured or generated by an MRI scanner, in which case the medical image 104 can be considered as an MRI scanned image. As yet another non-limiting example, the medical image 104 can be captured or generated by an X-ray scanner, in which case the medical image 104 can be considered as an X-ray scanned image. As even another non-limiting example, the medical image 104 can be captured or generated by an ultrasound scanner, in which case the medical image 104 can be considered as an ultrasound scanned image. As still another non-limiting example, the medical image 104 can be captured or generated by a PET scanner, in which case the medical image 104 can be considered as a PET scanned image. In various instances, the medical image 104 can have undergone any suitable image reconstruction techniques, such as filtered back projection.

In various aspects, the medical image 104 can visually depict or illustrate an anatomical structure 106 of any suitable medical patient (e.g., human, animal, or otherwise). In various instances, the anatomical structure 106 can be any suitable bodily organ of the medical patient, any suitable bodily tissue of the medical patient, any suitable body part of the medical patient, any suitable bodily fluid of the medical patient, any suitable bodily cavity of the medical patient, any suitable surgical implant (e.g., medical tubing, medical stitches, medical stents, pacemakers, medical rods, medical plates, medical screws), any suitable pathology thereof, or any suitable portion thereof.

In various aspects, it can be desired to localize the anatomical structure 106 within the medical image 104. In other words, it can be desired to determine specifically where the anatomical structure 106 is located within the medical image 104. In various cases, the ensembled visual querying system 102 can facilitate such localization.

In various embodiments, the ensembled visual querying system 102 can comprise a processor 108 (e.g., computer processing unit, microprocessor) and a non-transitory computer-readable memory 110 that is operably or operatively or communicatively connected or coupled to the processor 108. The non-transitory computer-readable memory 110 can store computer-executable instructions which, upon execution by the processor 108, can cause the processor 108 or other components of the ensembled visual querying system 102 (e.g., access component 112, example component 114, localization component 116, display component 118) to perform one or more acts. In various embodiments, the non-transitory computer-readable memory 110 can store computer-executable components (e.g., access component 112, example component 114, localization component 116, display component 118), and the processor 108 can execute the computer-executable components.

In various embodiments, the ensembled visual querying system 102 can comprise an access component 112. In various aspects, the access component 112 can electronically receive or otherwise electronically access the medical image 104. In various instances, the access component 112 can electronically retrieve the medical image 104 from any suitable centralized or decentralized data structures (not shown) or from any suitable centralized or decentralized computing devices (not shown). As a non-limiting example, the access component 112 can electronically retrieve the medical image 104 from whatever medical imaging modality (e.g., CT scanner, MRI scanner, X-ray scanner, ultrasound scanner, PET scanner) creates or generates the medical image 104. In any case, the access component 112 can electronically obtain or access the medical image 104, such that other components of the ensembled visual querying system 102 can electronically interact with the medical image 104.

In various embodiments, the ensembled visual querying system 102 can comprise an example component 114. In various aspects, the example component 114 can access a plurality of example medical images and a respectively corresponding plurality of user-provided clicks, where each user-provided click flags or otherwise marks an instantiation of the anatomical structure 106 within a respective one of the plurality of example medical images.

In various embodiments, the ensembled visual querying system 102 can comprise a localization component 116. In various instances, the localization component 116 can generate, via patch-wise execution of an embedder neural network on the medical image and on the plurality of example medical images, an ensembled heat map that indicates where the anatomical structure 106 is likely to be within the medical image 104.

In various embodiments, the ensembled visual querying system 102 can comprise a display component 118. In various cases, the display component 118 can transmit the ensembled heat map to any suitable computing device or can visually render the ensembled heat map on any suitable computer screen.

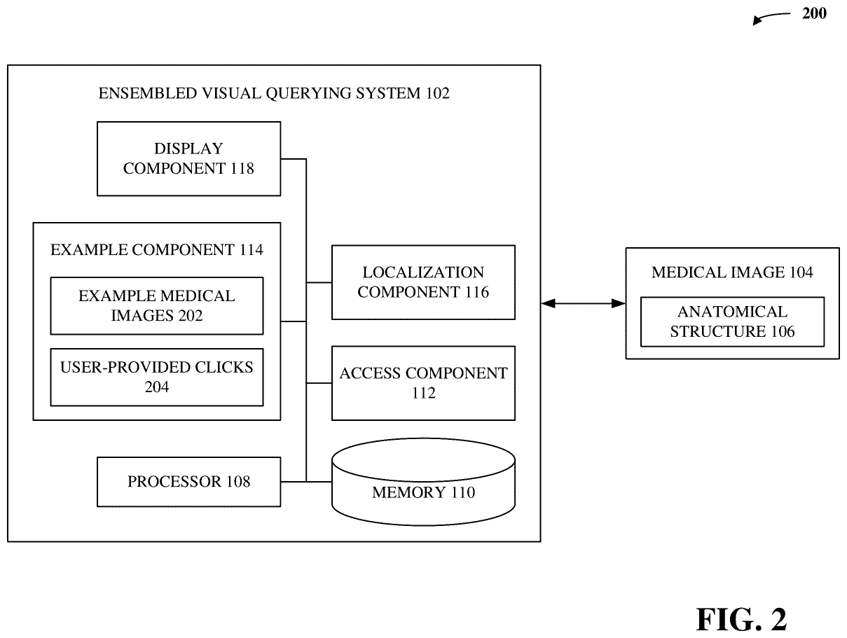

FIG. 2 illustrates a block diagram of an example, non-limiting system 200 including a plurality of example medical images and corresponding user-provided clicks that can facilitate ensembled querying of example images via deep learning embeddings in accordance with one or more embodiments described herein. As shown, the system 200 can, in some cases, comprise the same components as the system 100, and can further comprise a plurality of example medical images 202 and a plurality of user-provided clicks 204.

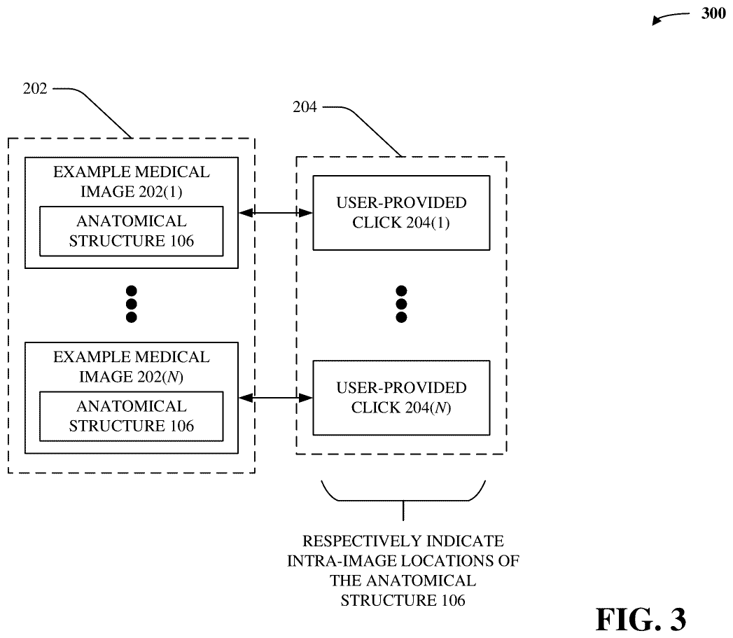

In various aspects, the example component 114 can electronically access, from any suitable centralized or decentralized data structures or computing devices, the plurality of example medical images 202 and the plurality of user-provided clicks 204. In various instances, each of the plurality of example medical images 202 can be a medical image that is different, distinct, or otherwise not identical to the medical image 104 but that nevertheless illustrates some instantiation or version of the anatomical structure 106. In various cases, each of the plurality of user-provided clicks 204 can be considered as an intra-image location (e.g., as an intra-image coordinate indicator) at which an instantiation or version of the anatomical structure 106 is known or deemed to be located within a respective one of the plurality of example medical images 202. Non-limiting aspects are described with respect to FIG. 3.

FIG. 3 illustrates an example, non-limiting block diagram 300 showing the plurality of example medical images 202 and the plurality of user-provided clicks 204 in accordance with one or more embodiments described herein.

In various embodiments, the plurality of example medical images 202 can comprise n images, for any suitable positive integer n≥2: an example medical image 202(1) to an example medical image 202(n). In various aspects, each of the plurality of example medical images 202 can exhibit the same format, size, or dimensionality as the medical image 104. As a non-limiting example, suppose that the medical image 104 is an x-by-y array of pixels. In such case, each of the plurality of example medical images 202 can likewise be an x-by-y array of pixels. As another non-limiting example, suppose that the medical image 104 is an x-by-y-by-z array of voxels. In such case, each of the plurality of example medical images 202 can likewise be an x-by-y-by-z array of voxels.

In various instances, each of the plurality of example medical images 202 can be different or otherwise non-identical to the medical image 104, but each of the plurality of example medical images 202 can nevertheless be known or deemed to depict or illustrate some instantiation, version, or manifestation of the anatomical structure 106. In some cases, each of the plurality of example medical images 202 can correspond to a respective medical patient that is different from or otherwise not related to the medical patient whose anatomy is pictured in the medical image 104, and each of the plurality of example medical images 202 can be known or deemed to depict or illustrate whatever instantiation, version, or manifestation of the anatomical structure 106 that belongs to its respective medical patient.

As a non-limiting example, suppose that the medical image 104 depicts a cerebral brain tumor of a given medical patient. In such case, the example medical image 202(1) can be known or deemed to depict a cerebral brain tumor of some different medical patient, and the example medical image 202(n) can be known or deemed to depict a cerebral brain tumor of yet some other medical patient. Note that, because each of the plurality of example medical images 202 can uniquely correspond to a respective medical patient, it can be the case that physical attributes, properties, or characteristics of the anatomical structure 106 vary across the plurality of example medical images 202. For instance, the anatomical structure 106 can be differently sized, differently shaped, or differently shaded in the medical image 104 than in the example medical image 202(1); the anatomical structure 106 can be differently sized, differently shaped, or differently shaded in the example medical image 202(1) than in an example medical image 202(2) (not shown); the anatomical structure 106 can be differently sized, differently shaped, or differently shaded in the example medical image 202(2) than in an example medical image 202(3) (not shown); and, continuing in this fashion, the anatomical structure 106 can be differently sized, differently shaped, or differently shaded in an example medical image 202(n−1) (not shown) than in the example medical image 202(n).

In any case, each of the plurality of example medical images 202 can be known or deemed to illustrate a respective instantiation of the anatomical structure 106.

In various aspects, the plurality of user-provided clicks 204 can respectively correspond (e.g., in one-to-one fashion) to the plurality of example medical images 202. Thus, since the plurality of example medical images 202 can comprise n images, the plurality of user-provided clicks 204 can comprise n clicks: a user-provided click 204(1) to a user-provided click 204(n). In various instances, each of the plurality of user-provided clicks 204 can be any suitable electronic data that indicates an intra-image location or spatial coordinate, within a respective one of the plurality of example medical images 202, at which at least part of an instantiation of the anatomical structure 106 is known or deemed to be located. As a non-limiting example, the user-provided click 204(1) can correspond to the example medical image 202(1). Accordingly, the user-provided click 204(1) can indicate, mark, or otherwise flag a specific spatial coordinate or point inside of the example medical image 202(1) at which the anatomical structure 106 is known to be located or positioned. As another non-limiting example, the user-provided click 204(n) can correspond to the example medical image 202(n). So, the user-provided click 204(n) can indicate, mark, or otherwise flag a particular spatial coordinate or point inside of the example medical image 202(n) at which the anatomical structure 106 is known to be located or positioned.

In various aspects, the plurality of user-provided clicks 204 can be supplied by a medical professional or subject matter expert. For instance, such medical professional or subject matter expert can visually inspect each of the plurality of example medical images 202, and the medical professional or subject matter expert can generate the plurality of user-provided clicks 204 by interacting with any suitable human-computer interface devices (e.g., by clicking on a computer mouse or touchscreen).

In various instances, n can be rather small. As a non-limiting example, it can be the case that 2≤n≤5. As another non-limiting example, it can be the case that 2≤n≤10.

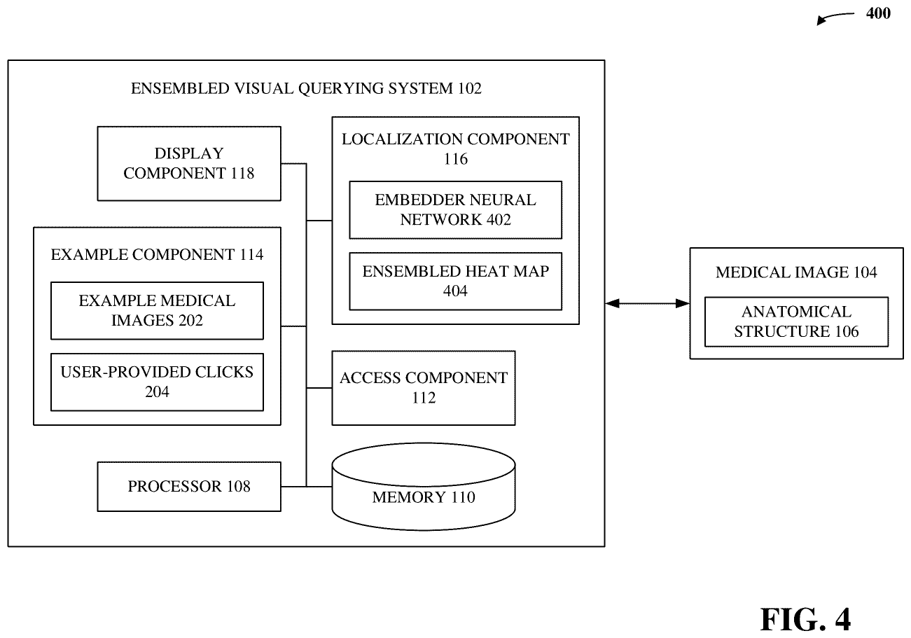

FIG. 4 illustrates a block diagram of an example, non-limiting system 400 including an embedder neural network and an ensembled heat map that can facilitate ensembled querying of example images via deep learning embeddings in accordance with one or more embodiments described herein. As shown, the system 400 can, in some cases, comprise the same components as the system 200, and can further comprise an embedder neural network 402 and an ensembled heat map 404.

In various aspects, the localization component 116 can electronically store, electronically maintain, electronically control, or otherwise electronically access the embedder neural network 402. In various aspects, the embedder neural network 402 can be any suitable artificial neural network that can have or otherwise exhibit any suitable internal architecture. For instance, the embedder neural network 402 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

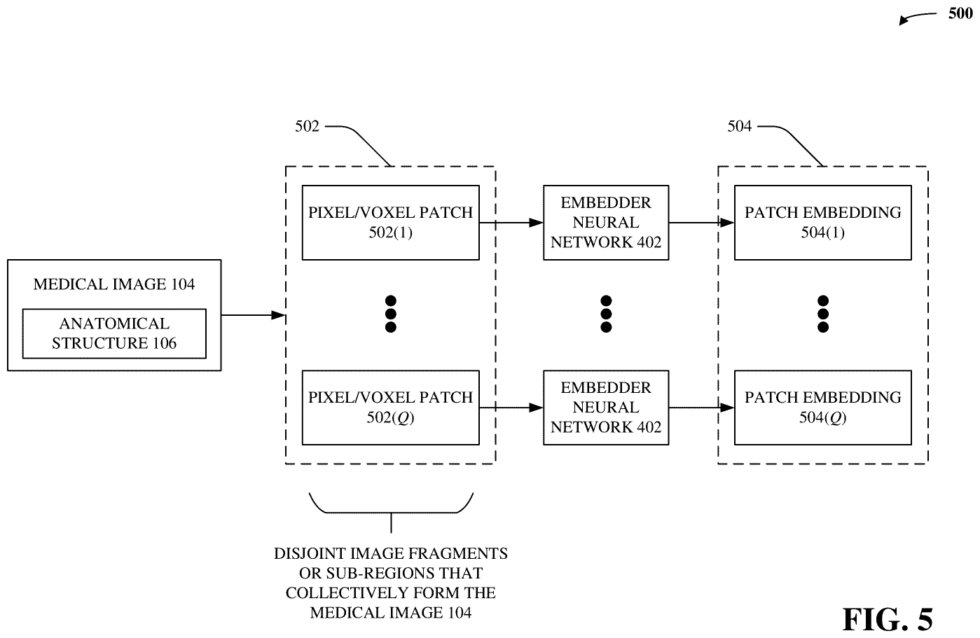

Regardless of its internal architecture, the embedder neural network 402 can be configured, as described herein, to generate embeddings of inputted patches of pixels (or voxels). In various instances, as described herein, the localization component 116 can electronically generate an ensembled heat map 404, by executing the embedder neural network 402 in patch-wise fashion on the medical image 104 and on the plurality of example medical images 202. In various cases, as described herein, the ensembled heat map 404 can show or otherwise indicate where the anatomical structure 106 is located or positioned within the medical image 104. Non-limiting aspects are described with respect to FIGS. 5-9.

FIGS. 5-9 illustrate example, non-limiting block diagrams 500, 600, 700, 800, and 900 showing how the embedder neural network 402 can be implemented to generate the ensembled heat map 404 in accordance with one or more embodiments described herein.

First, consider FIG. 5. In various aspects, the localization component 116 can decompose or otherwise divide the medical image 104 into a set of pixel/voxel patches 502. In various instances, the set of pixel/voxel patches 502 can comprise q patches, for any suitable positive integer q: a pixel/voxel patch 502(1) to a pixel/voxel patch 502(q). In various cases, the set of pixel/voxel patches 502 can be regularly-repeating, disjoint fragments or regions into which the medical image 104 can be broken or divided. As a non-limiting example, suppose that the medical image 104 is an x-by-y array of pixels. In such case, each of the set of pixel/voxel patches 502 can be a distinct a-by-b tile of adjacent pixels from the medical image 104, for any suitable positive integers a and b such that

$$ab = \frac{xy}{q} ,$$

where the union of all of such a-by-b tiles is equal to the medical image 104, and where none of such a-by-b tiles overlap with each other. As another non-limiting example, suppose that the medical image 104 is an x-by-y-by-z array of voxels. In such case, each of the set of pixel/voxel patches 502 can be a distinct a-by-b-by-c block of adjacent voxels from the medical image 104, for any suitable positive integers a, b, and c such that

$$abc = \frac{xyz}{q} ,$$

where the union of all of such a-by-b-by-c blocks is equal to the medical image 104, and where none of such a-by-b-by-c blocks overlap with each other.

In various aspects, the localization component 116 can execute the embedder neural network 402 on each of the set of pixel/voxel patches 502. In various instances, such execution can yield a set of patch embeddings 504.

As a non-limiting example, the localization component 116 can feed the pixel/voxel patch 502(1) to an input layer of the embedder neural network 402. In various aspects, the pixel/voxel patch 502(1) can complete a forward pass through one or more hidden layers of the embedder neural network 402. In various instances, an output layer of the embedder neural network 402 can compute or otherwise calculate a patch embedding 504(1), based on activation maps generated by the one or more hidden layers of the embedder neural network 402. In any case, the patch embedding 504(1) can be considered as a latent vector representation that the embedder neural network 402 believes or infers corresponds to the pixel/voxel patch 502(1). More specifically, the patch embedding 504(1) can be one or more scalars, one or more vectors, one or more matrices, one or more tensors, or any suitable combination thereof. In various aspects, the dimensionality of the patch embedding 504(1) (e.g., the total number of numerical elements within the patch embedding 504(1)) can be smaller (e.g., many orders of magnitude smaller, in some cases) than the dimensionality of the pixel/voxel patch 502(1). In various instances, despite its smaller dimensionality, the patch embedding 504(1) can nevertheless be considered as representing, albeit in hidden or non-apparent fashion, at least some substantive visual content that is depicted or illustrated by the pixel/voxel patch 502(1).

As another non-limiting example, the localization component 116 can feed the pixel/voxel patch 502(q) to the input layer of the embedder neural network 402. In various aspects, the pixel/voxel patch 502(q) can complete a forward pass through the one or more hidden layers of the embedder neural network 402. In various instances, the output layer of the embedder neural network 402 can compute or otherwise calculate a patch embedding 504(q), based on activation maps generated by the one or more hidden layers of the embedder neural network 402. In any case, just as above, the patch embedding 504(q) can be considered as a latent vector representation (e.g., having the same format, size, or dimensionality as the patch embedding 504(1)) that the embedder neural network 402 believes or infers corresponds to the pixel/voxel patch 502(q). So, the dimensionality of the patch embedding 504(q) (e.g., the total number of numerical elements within the patch embedding 504(q)) can be smaller (e.g., many orders of magnitude smaller, in some cases) than the dimensionality of the pixel/voxel patch 502(q). However, despite its smaller dimensionality, the patch embedding 504(q) can nevertheless be considered as representing, albeit in hidden or non-apparent fashion, at least some substantive visual content that is depicted or illustrated by the pixel/voxel patch 502(q).

In various aspects, the patch embedding 504(1) to the patch embedding 504(q) can collectively be considered as forming the set of patch embeddings 504.

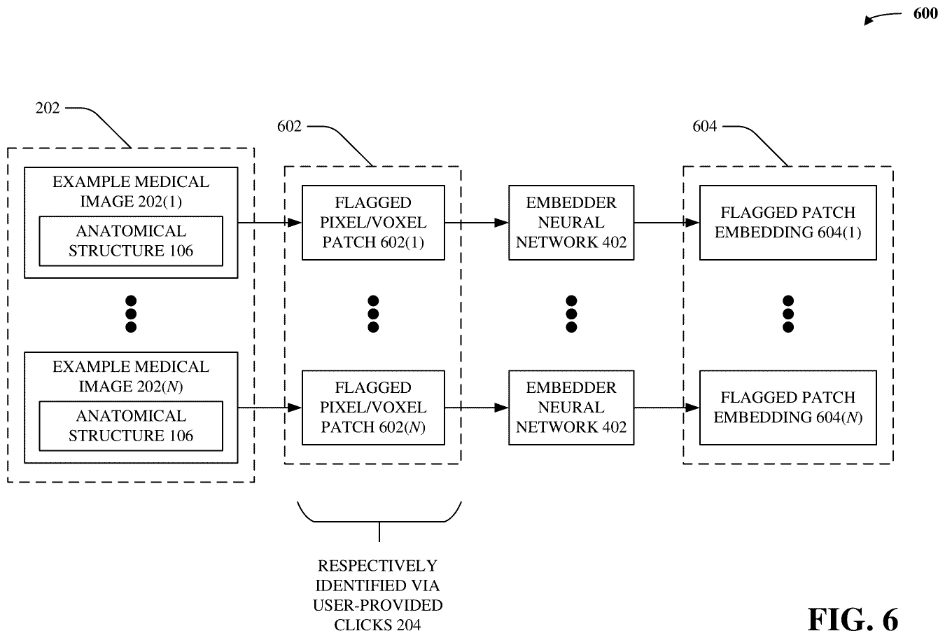

Now, consider FIG. 6. In various embodiments, the plurality of example medical images 202 can respectively correspond (e.g., in one-to-one fashion) to a plurality of flagged pixel/voxel patches 602. In various aspects, the plurality of flagged pixel/voxel patches 602 can be respectively indicated or identified by the plurality of user-provided clicks 204.

As a non-limiting example, the example medical image 202(1) can, just like the medical image 104, be decomposed, divided, or otherwise broken up into a total of q pixel (or voxel) patches (not shown). For instance, if the example medical image 202(1) is an x-by-y pixel array, then the example medical image 202(1) can be decomposed into q distinct a-by-b tiles of adjacent pixels, for any suitable positive integers

$$ab = \frac{xy}{q} .$$

As another instance, if the example medical image 202(1) is an x-by-y-by-z voxel array, then the example medical image 202(1) can be decomposed into q distinct a-by-b-by-c blocks of adjacent voxels, for any suitable positive integers

$$abc = \frac{xyz}{q} .$$

In any case, the user-provided click 204(1) can be located within (e.g., can indicate an intra-image spatial coordinate that is positioned inside of) a specific one of those q pixel (or voxel) patches. In various instances, that specific pixel (or voxel) patch of the example medical image 202(1) can be considered as being marked, indicated, or otherwise flagged by the user-provided click 204(1). Accordingly, that specific pixel (or voxel) patch can be referred to as a flagged pixel/voxel patch 602(1). Because the flagged pixel/voxel patch 602(1) can be indicated or marked by the user-provided click 204(1), the flagged pixel/voxel patch 602(1) can be considered as a specific tile or block of the example medical image 202(1) that is known or deemed to depict an instantiation of the anatomical structure 106.

As another non-limiting example, the example medical image 202(n) can, just like the medical image 104, be decomposed, divided, or otherwise broken up into a total of q pixel (or voxel) patches (not shown) (e.g., can be decomposed into q distinct a-by-b tiles of adjacent pixels or q distinct a-by-b-by-c blocks of adjacent voxels, as appropriate). In any case, the user-provided click 204(1) can be located within a specific one of those q pixel (or voxel) patches, and so that specific pixel (or voxel) patch can be considered as being marked, indicated, or otherwise flagged by the user-provided click 204(n). So, that specific pixel (or voxel) patch can be referred to as a flagged pixel/voxel patch 602(n). Because the flagged pixel/voxel patch 602(n) can be indicated or marked by the user-provided click 204(n), the flagged pixel/voxel patch 602(n) can be considered as a specific tile or block of the example medical image 202(n) that is known or deemed to depict an instantiation of the anatomical structure 106.

In various cases, the flagged pixel/voxel patch 602(1) to the flagged pixel/voxel patch 602(n) can collectively be considered as forming the set of flagged pixel/voxel patches 602.

In various aspects, the localization component 116 can execute the embedder neural network 402 on each of the set of flagged pixel/voxel patches 602. In various instances, such execution can yield a set of flagged patch embeddings 604.

As a non-limiting example, the localization component 116 can feed the flagged pixel/voxel patch 602(1) to the input layer of the embedder neural network 402. In various aspects, the flagged pixel/voxel patch 602(1) can complete a forward pass through the one or more hidden layers of the embedder neural network 402. In various instances, the output layer of the embedder neural network 402 can compute or otherwise calculate a flagged patch embedding 604(1), based on activation maps generated by the one or more hidden layers of the embedder neural network 402. In any case, the flagged patch embedding 604(1) can be considered as a latent vector representation (e.g., having the same format, size, or dimensionality as each of the set of patch embeddings 504) that the embedder neural network 402 believes or infers corresponds to the flagged pixel/voxel patch 602(1). So, the flagged patch embedding 604(1) can be considered as representing, albeit in hidden or non-apparent fashion, at least some substantive visual content that is depicted or illustrated by the flagged pixel/voxel patch 602(1).

As another non-limiting example, the localization component 116 can feed the flagged pixel/voxel patch 602(n) to the input layer of the embedder neural network 402. In various aspects, the flagged pixel/voxel patch 602(n) can complete a forward pass through the one or more hidden layers of the embedder neural network 402. In various instances, the output layer of the embedder neural network 402 can compute or otherwise calculate a flagged patch embedding 604(n), based on activation maps generated by the one or more hidden layers of the embedder neural network 402. In any case, the flagged patch embedding 604(n) can be considered as a latent vector representation (e.g., having the same format, size, or dimensionality as each of the set of patch embeddings 504) that the embedder neural network 402 believes or infers corresponds to the flagged pixel/voxel patch 602(n). Thus, the flagged patch embedding 604(n) can be considered as representing, albeit in hidden or non-apparent fashion, at least some substantive visual content that is depicted or illustrated by the flagged pixel/voxel patch 602(n).

In various aspects, the flagged patch embedding 604(1) to the flagged patch embedding 604(n) can collectively be considered as forming the set of flagged patching embeddings 604.

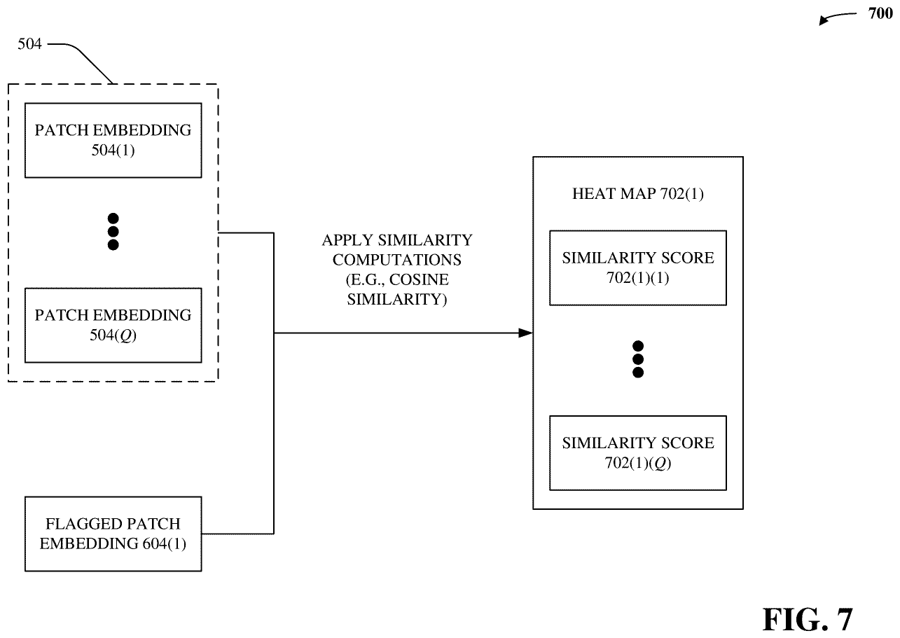

Next, consider FIG. 7. In various instances, the localization component 116 can generate a heat map 702(1), based on the set of patch embeddings 504 and based on the flagged patch embedding 604(1). More specifically, the localization component 116 can generate the heat map 702(1), by computing similarity scores between the flagged patch embedding 604(1) and each of the set of patch embeddings 504.

As a non-limiting example, the localization component 116 can compute a similarity score 702(1)(1) between the flagged patch embedding 604(1) and the patch embedding 504(1). In various aspects, the similarity score 702(1)(1) can be achieved by applying any suitable similarity measurement or computation between the flagged patch embedding 604(1) and the patch embedding 504(1). As a non-limiting example, the similarity score 702(1)(1) can be equal to, or otherwise based on, a cosine similarity computed between the flagged patch embedding 604(1) and the patch embedding 504(1). In any case, the similarity score 702(1)(1) can be a scalar whose magnitude indicates how similar or how dissimilar the flagged patch embedding 604(1) is from the patch embedding 504(1). Recall that the flagged patch embedding 604(1) can represent whatever visual content is depicted by the flagged pixel/voxel patch 602(1) of the example medical image 202(1). Since the flagged pixel/voxel patch 602(1) can be known or deemed to depict an instantiation of the anatomical structure 106, the flagged patch embedding 604(1) can be considered as representing that instantiation of the anatomical structure 106. Also recall that the patch embedding 504(1) can represent whatever visual content is depicted by the pixel/voxel patch 502(1) of the medical image 104. Therefore, the similarity score 702(1)(1) can be considered as indicating whether or not the visual content of the pixel/voxel patch 502(1) matches or is otherwise similar to whatever instantiation of the anatomical structure 106 is illustrated in the flagged pixel/voxel patch 602(1). As a non-limiting example, higher magnitudes (e.g., closer to 1) of the similarity score 702(1)(1) can indicate more similarity between the visual content of the pixel/voxel patch 502(1) and the instantiation of the anatomical structure 106 that is illustrated in the flagged pixel/voxel patch 602(1), whereas lower magnitudes (e.g., closer to 0) of the similarity score 702(1)(1) can indicate less similarity between the visual content of the pixel/voxel patch 502(1) and the instantiation of the anatomical structure 106 that is illustrated in the flagged pixel/voxel patch 602(1). Note that the similarity score 702(1)(1) can be computed, no matter the type or class of the anatomical structure 106.

As another non-limiting example, the localization component 116 can compute a similarity score 702(1)(q) between the flagged patch embedding 604(1) and the patch embedding 504(q). Just as above, the similarity score 702(1)(q) can be achieved by applying any suitable similarity measurement or computation between the flagged patch embedding 604(1) and the patch embedding 504(q). As a non-limiting example, the similarity score 702(1)(q) can be equal to, or otherwise based on, a cosine similarity computed between the flagged patch embedding 604(1) and the patch embedding 504(q). In any case, the similarity score 702(1)(q) can be a scalar whose magnitude indicates how similar or how dissimilar the flagged patch embedding 604(1) is from the patch embedding 504(q). Recall that the flagged patch embedding 604(1) can represent whatever instantiation of the anatomical structure 106 is depicted by the flagged pixel/voxel patch 602(1) of the example medical image 202(1). Also recall that the patch embedding 504(q) can represent whatever visual content is depicted by the pixel/voxel patch 502(q) of the medical image 104. Thus, the similarity score 702(1)(q) can be considered as indicating whether or not the visual content of the pixel/voxel patch 502(q) matches, is similar to, or is dissimilar to whatever instantiation of the anatomical structure 106 is illustrated in the flagged pixel/voxel patch 602(1). Again, note that the similarity score 702(1)(q) can be computed, no matter the type or class of the anatomical structure 106.

In various cases, the similarity score 702(1)(1) to the similarity score 702(1)(q) can collectively be considered as forming the heat map 702(1). Accordingly, the heat map 702(1) can be considered as a patch-wise array that indicates which of the set of pixel/voxel patches 502 are more similar or are less similar to the flagged pixel/voxel patch 602(1). In other words, the heat map 702(1) can be considered as a patch-wise array that indicates which of the set of pixel/voxel patches 502 are more likely or less likely to depict whatever instantiation of the anatomical structure 106 is shown in the example medical image 202(1).

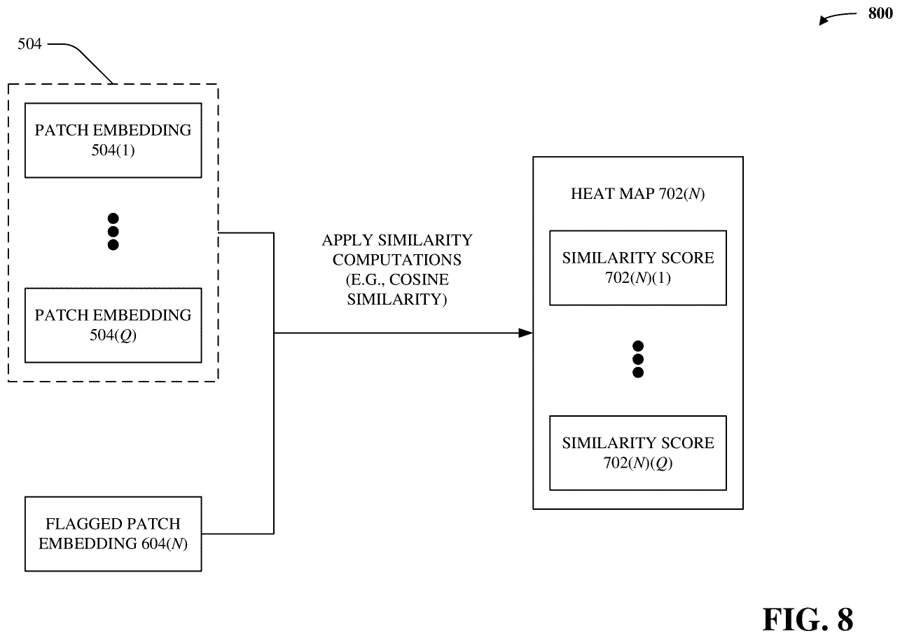

Now, consider FIG. 8. In various instances, the localization component 116 can generate a heat map 702(n), based on the set of patch embeddings 504 and based on the flagged patch embedding 604(n). More specifically, the localization component 116 can generate the heat map 702(n), by computing similarity scores between the flagged patch embedding 604(n) and each of the set of patch embeddings 504.

As a non-limiting example, the localization component 116 can compute a similarity score 702(n)(1) between the flagged patch embedding 604(n) and the patch embedding 504(1). In various aspects, the similarity score 702(n)(1) can be achieved by applying any suitable similarity measurement or computation between the flagged patch embedding 604(n) and the patch embedding 504(1). As a non-limiting example, the similarity score 702(n)(1) can be equal to, or otherwise based on, a cosine similarity computed between the flagged patch embedding 604(n) and the patch embedding 504(1). In any case, the similarity score 702(n)(1) can be a scalar whose magnitude indicates how similar or how dissimilar the flagged patch embedding 604(n) is from the patch embedding 504(1). Recall that the flagged patch embedding 604(n) can represent whatever visual content is depicted by the flagged pixel/voxel patch 602(n) of the example medical image 202(n). Since the flagged pixel/voxel patch 602(n) can be known or deemed to depict an instantiation of the anatomical structure 106, the flagged patch embedding 604(n) can be considered as representing that instantiation of the anatomical structure 106. Also recall that the patch embedding 504(1) can represent whatever visual content is depicted by the pixel/voxel patch 502(1) of the medical image 104. Therefore, the similarity score 702(n)(1) can be considered as indicating whether or not the visual content of the pixel/voxel patch 502(1) matches or is otherwise similar to whatever instantiation of the anatomical structure 106 is illustrated in the flagged pixel/voxel patch 602(n). As above, note that the similarity score 702(n)(1) can be computed, no matter the type or class of the anatomical structure 106.

As another non-limiting example, the localization component 116 can compute a similarity score 702(n)(q) between the flagged patch embedding 604(n) and the patch embedding 504(q). Just as above, the similarity score 702(n)(q) can be achieved by applying any suitable similarity measurement or computation between the flagged patch embedding 604(n) and the patch embedding 504(q). As a non-limiting example, the similarity score 702(n)(q) can be equal to, or otherwise based on, a cosine similarity computed between the flagged patch embedding 604(n) and the patch embedding 504(q). In any case, the similarity score 702(n)(q) can be a scalar whose magnitude indicates how similar or how dissimilar the flagged patch embedding 604(n) is from the patch embedding 504(q). Recall that the flagged patch embedding 604(n) can represent whatever instantiation of the anatomical structure 106 is depicted by the flagged pixel/voxel patch 602(n) of the example medical image 202(n). Also recall that the patch embedding 504(q) can represent whatever visual content is depicted by the pixel/voxel patch 502(q) of the medical image 104. Thus, the similarity score 702(n)(q) can be considered as indicating whether or not the visual content of the pixel/voxel patch 502(q) matches, is similar to, or is dissimilar to whatever instantiation of the anatomical structure 106 is illustrated in the flagged pixel/voxel patch 602(n). Yet again, note that the similarity score 702(n)(q) can be computed, no matter the type or class of the anatomical structure 106.

In various cases, the similarity score 702(n)(1) to the similarity score 702(n)(q) can collectively be considered as forming the heat map 702(n). Accordingly, the heat map 702(n) can be considered as a patch-wise array that indicates which of the set of pixel/voxel patches 502 are more similar or are less similar to the flagged pixel/voxel patch 602(n). In other words, the heat map 702(n) can be considered as a patch-wise array that indicates which of the set of pixel/voxel patches 502 are more likely or less likely to depict whatever instantiation of the anatomical structure 106 is shown in the example medical image 202(n).

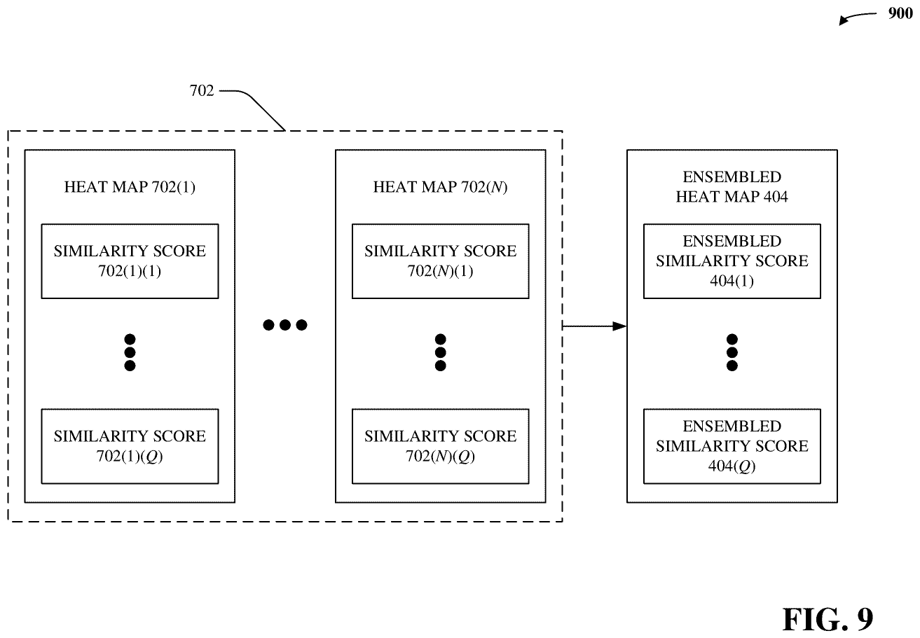

Next, consider FIG. 9. In various aspects, the heat map 702(1) to the heat map 702(n) can be considered as collectively forming a plurality of heat maps 702. In various instances, the localization component 116 can generate the ensembled heat map 404, by aggregating the plurality of heat maps 702 together.

As a non-limiting example, the localization component 116 can aggregate the similarity score 702(1)(1) to the similarity score 702(n)(1) together, thereby yielding an ensembled similarity score 404(1). For instance, the ensembled similarity score 404(1) can be equal to, or otherwise based on, a sum or an average of the similarity score 702(1)(1) to the similarity score 702(n)(1). In any case, because the similarity score 702(1)(1) to the similarity score 702(n)(1) can all correspond to the pixel/voxel patch 502(1), the ensembled similarity score 404(1) can likewise correspond to the pixel/voxel patch 502(1). Thus, the ensembled similarity score 404(1) can be a scalar whose magnitude can be interpreted as indicating a likelihood or probability that the pixel/voxel patch 502(1) depicts the anatomical structure 106.

As another non-limiting example, the localization component 116 can aggregate the similarity score 702(1)(q) to the similarity score 702(n)(q) together, thereby yielding an ensembled similarity score 404(q). For instance, the ensembled similarity score 404(q) can be equal to, or otherwise based on, a sum or an average of the similarity score 702(1)(q) to the similarity score 702(n)(q). In any case, because the similarity score 702(1)(q) to the similarity score 702(n)(q) can all correspond to the pixel/voxel patch 502(q), the ensembled similarity score 404(q) can likewise correspond to the pixel/voxel patch 502(q). So, the ensembled similarity score 404(q) can be a scalar whose magnitude can be interpreted as indicating a likelihood or probability that the pixel/voxel patch 502(q) depicts the anatomical structure 106.

Accordingly, the ensembled heat map 404 can indicate at what locations (if any) within the medical image 104 that the anatomical structure 106 is likely to be located. Indeed, note that, in some cases, it can be possible that the medical image 104 does not depict or illustrate the anatomical structure 106 at all. In such cases, the ensembled heat map 404 can indicate low similarity scores for all of the set of pixel/voxel patches 502.

Note that the ensembled heat map 404 can be considered as being robust or reliable, due to being an aggregation of the plurality of heat maps 702. Indeed, if any of the plurality of example medical images 202 exhibits visual artifacts or is otherwise visually unusual (e.g., is distorted, has an unexpected brightness/contrast), then whichever one of the plurality of heat maps 702 that is generated based on that example medical image can be incorrect, inaccurate, or unreliable. However, it can be unlikely for a majority of the plurality of example medical images 202 to exhibit visual artifacts or to otherwise be visually unusual. Thus, it can be commensurately unlikely for a majority of the plurality of heat maps 702 to be incorrect, inaccurate, or unreliable. Accordingly, those few (if any) of the plurality of heat maps 702 that are incorrect, inaccurate, or unreliable can be considered as being averaged-out when the plurality of heat maps 702 are aggregated into the ensembled heat map 404.

In various embodiments, the display component 118 can electronically perform any suitable actions based on the ensembled heat map 404. As a non-limiting example, the display component 118 can electronically transmit the ensembled heat map 404 to any other suitable computing device (not shown). As another non-limiting example, the display component 118 can electronically render the ensembled heat map 404 on any suitable electronic display (not shown), such as a computer screen or computer monitor.

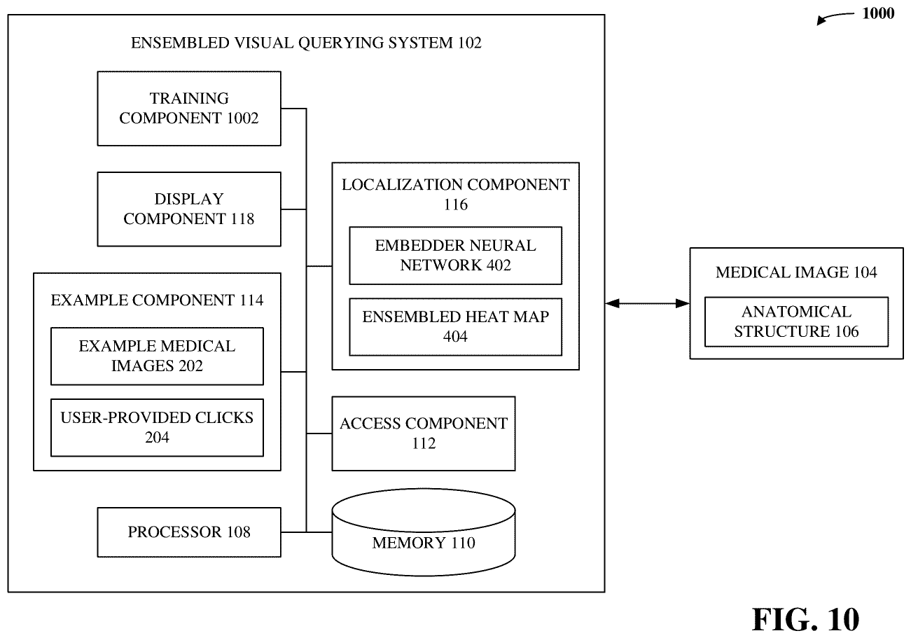

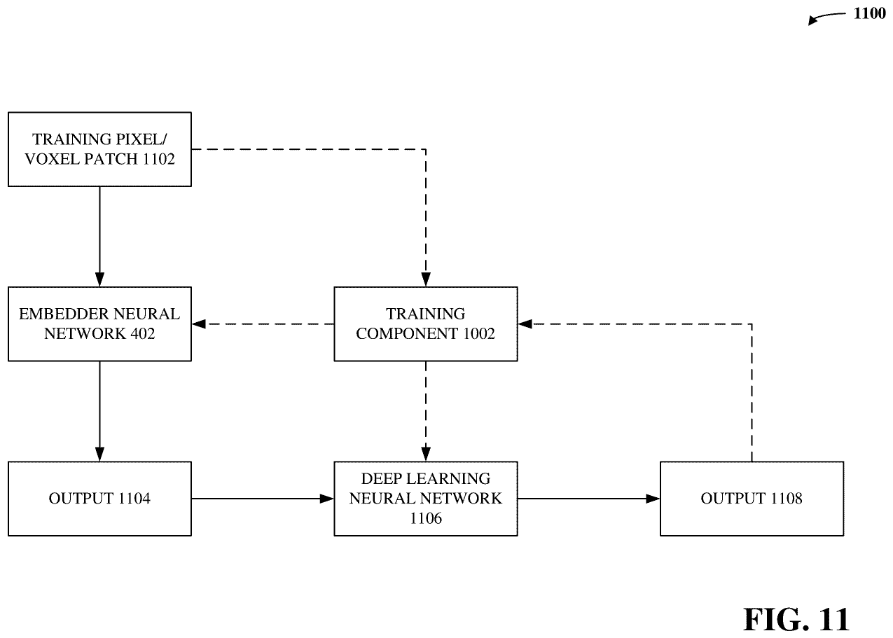

In order for the various embeddings described herein to be accurate, the embedder neural network 402 can first undergo training, as described with respect to FIGS. 10-11.

FIG. 10 illustrates a block diagram of an example, non-limiting system 1000 including a training component that can facilitate ensembled querying of example images via deep learning embeddings in accordance with one or more embodiments described herein. As shown, the system 1000 can, in some cases, comprise the same components as the system 400, and can further comprise a training component 1002.

In various aspects, the training component 1002 can train the embedder neural network 402 in unsupervised fashion. As a non-limiting example, the training component 1002 can train the embedder neural network 402 as part of an encoder-decoder deep learning pipeline. Various non-limiting aspects are shown with respect to FIG. 11.

FIG. 11 illustrates an example, non-limiting block diagram 1100 showing how the embedder neural network 402 can be trained in accordance with one or more embodiments described herein.

In various embodiments, there can be a deep learning neural network 1106. In various aspects, the deep learning neural network 1106 can be any suitable artificial neural network that can have or otherwise exhibit any suitable internal architecture. For instance, the deep learning neural network 1106 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

Regardless of its internal architecture, the deep learning neural network 1106 can be configured to perform an inverse function as the embedder neural network 402. That is, while the embedder neural network 402 can be configured to generate embeddings (e.g., latent vectors) from inputted pixel (or voxel) patches, the deep learning neural network 1106 can instead be configured to generate pixel (or voxel) patches based on inputted embeddings.

In various aspects, the training component 1002 can leverage the deep learning neural network 1106 in order to train the embedder neural network 402, as described herein.

At the start of such training, the training component 1002 can initialize in any suitable fashion (e.g., random initialization) the trainable internal parameters (e.g., weight matrices, bias values, convolutional kernels) of the embedder neural network 402 and of the deep learning neural network 1106.

In various aspects, the training component 1002 can select, obtain, or otherwise access a training pixel/voxel patch 1102. In various instances, the training pixel/voxel patch 1102 can exhibit the same format, size, or dimensionality as each of the set of pixel/voxel patches 502, and the training pixel/voxel patch 1102 can come from any suitable image whatsoever (can even come from a non-medical image).

In any case, the embedder neural network 402 and the deep learning neural network 1106 can be serially coupled or arranged, such that the embedder neural network 402 is upstream of the deep learning neural network 1106; equivalently, the deep learning neural network 1106 can be downstream of the embedder neural network 402.

In various aspects, the training component 1002 can execute the embedder neural network 402 on the training pixel/voxel patch 1102. In various instances, this can cause the embedder neural network 402 to produce an output 1104. More specifically, the training component 1002 can feed the training pixel/voxel patch 1102 to the input layer of the embedder neural network 402. In various cases, the training pixel/voxel patch 1102 can complete a forward pass through the one or more hidden layers of the embedder neural network 402. Accordingly, the output layer of the embedder neural network 402 can compute or calculate the output 1104 based on activation maps produced by the one or more hidden layers of the embedder neural network 402.

Note that, in various cases, the format, size, or dimensionality of the output 1104 can be controlled or otherwise determined by the number, arrangement, or sizes of neurons or of other internal parameters (e.g., convolutional kernels) that are contained in or that otherwise make up the output layer (or any other layers) of the embedder neural network 402. Thus, the output 1104 can be forced to have any desired format, size, or dimensionality by adding, removing, or otherwise adjusting neurons or other internal parameters to, from, or within the output layer (or any other layers) of the embedder neural network 402. Accordingly, in various aspects, the output 1104 can be forced to have a smaller (e.g., in some cases, one or more orders of magnitude smaller) format, size, or dimensionality than the training pixel/voxel patch 1102. In such case, the output 1104 can be considered as an embedding (e.g., a latent vector representation) that the embedder neural network 402 has predicted or inferred corresponds to the training pixel/voxel patch 1102.

Additionally, note that, if the embedder neural network 402 has so far undergone no or little training, then the output 1104 can be highly inaccurate. In other words, the output 1104 can fail to be a correct or proper embedding of the training pixel/voxel patch 1102 (e.g., can fail to properly encode or represent substantive visual content of the training pixel/voxel patch 1102).

In various aspects, the training component 1002 can execute the deep learning neural network 1106 on the output 1104. In various instances, this can cause the deep learning neural network 1106 to produce an output 1108. In particular, the training component 1002 can feed the output 1104 to an input layer of the deep learning neural network 1106. In various cases, the output 1104 can complete a forward pass through one or more hidden layers of the deep learning neural network 1106. Accordingly, an output layer of the deep learning neural network 1106 can compute or calculate the output 1108 based on activation maps produced by the one or more hidden layers of the deep learning neural network 1106.

As above, note that the format, size, or dimensionality of the output 1108 can be controlled or otherwise determined by the number, arrangement, or sizes of neurons or other internal parameters (e.g., convolutional kernels) that are contained in or that otherwise make up the output layer (or any other layers) of the deep learning neural network 1106. Thus, the output 1108 can be forced to have any desired format, size, or dimensionality by adding, removing, or otherwise adjusting neurons or other internal parameters to, from, or within the output layer (or any other layers) of the deep learning neural network 1106. Accordingly, in various aspects, the output 1108 can be forced to have the same format, size, or dimensionality as the training pixel/voxel patch 1102. In such case, the output 1108 can be considered as an approximation or recreation of the training pixel/voxel patch 1102, as predicted or inferred by the deep learning neural network 1106.

Additionally, and just as above, note that, if the deep learning neural network 1106 has so far undergone no or little training, then the output 1108 can be highly inaccurate. That is, the output 1108 can be very different from the training pixel/voxel patch 1102.

In various aspects, the training component 1002 can compute an error or loss (e.g., mean absolute error, mean squared error, cross-entropy error) between the output 1108 and the training pixel/voxel patch 1102. In various instances, as shown, the training component 1002 can incrementally update the trainable internal parameters of both the embedder neural network 402 and of the deep learning neural network 1106, by performing backpropagation (e.g., stochastic gradient descent) driven by the computed error or loss. In other words, the embedder neural network 402 and the deep learning neural network 1106 can be considered as collectively forming an encoder-decoder deep learning pipeline. In such pipeline, the embedder neural network 402 can be considered as the encoder, whereas the deep learning neural network 1106 can be considered as the decoder.

In various cases, the training component 1002 can repeat the above-described training procedure for any suitable number of training pixel/voxel patches (e.g., for any suitable number of instances of 1102). This can ultimately cause the trainable internal parameters of the embedder neural network 402 to become iteratively optimized for accurately generating embeddings based on inputted pixel (or voxel) patches, and this can also ultimately cause the trainable internal parameters of the deep learning neural network 1106 to become iteratively optimized for accurately recreating pixel (or voxel) patches based on inputted embeddings. In various aspects, the training component 1002 can implement any suitable training batch sizes, any suitable training termination criterion, or any suitable error, loss, or objective function when training the embedder neural network 402 and the deep learning neural network 1106.

Although the above discussion mainly describes the embedder neural network 402 as being trained in an encoder-decoder deep learning pipeline, this is a mere non-limiting example for ease of illustration and explanation. In various other embodiments, the training component 1002 can train the embedder neural network 402 in any other suitable unsupervised or self-supervised fashion. As a non-limiting example, the training component 1002 can train the embedder neural network 402 via any suitable DINO technique (e.g., self-distillation-with-no-labels). In yet other embodiments, the embedder neural network 402 can be an encoder from any suitable pre-trained vision transformer. As a non-limiting example, the embedder neural network 402 can be an encoder from any suitable pre-trained Segment Anything Model (SAM) architecture or pipeline, such as an encoder from a SonoSAM model or from a MedSAM model. As another non-limiting example, the embedder neural network 402 can be an encoder from any suitable pre-trained Contrastive Language-Image Pre-training (CLIP) architecture or pipeline, such as an encoder from a MedCLIP model.

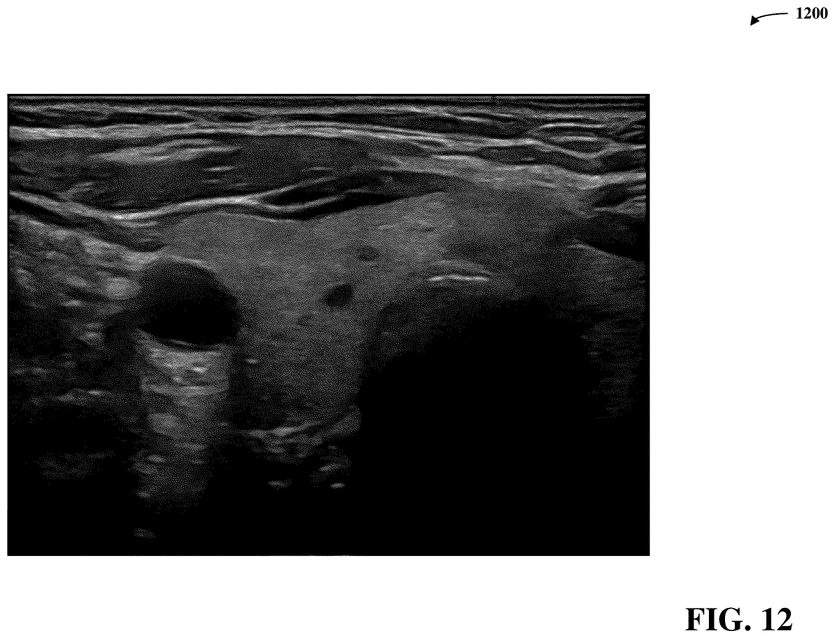

FIGS. 12-17 illustrate example, non-limiting experimental results in accordance with one or more embodiments described herein.

FIG. 12 shows an ultrasound image 1200 which depicts an anatomy of a first medical patient. It can be desired to localize both a common carotid artery and a thyroid within the ultrasound image 1200.

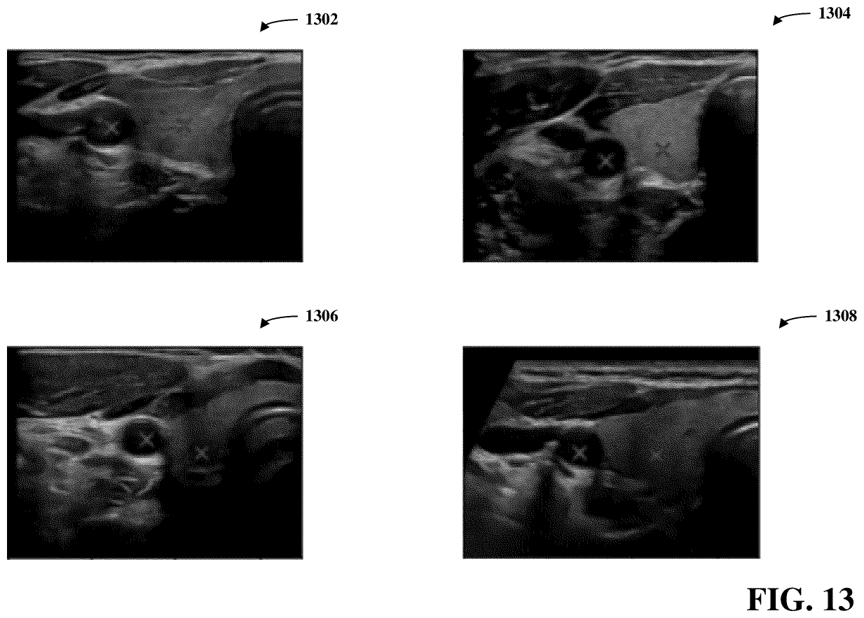

FIG. 13 shows four example ultrasound images: an example ultrasound image 1302; an example ultrasound image 1304; an example ultrasound image 1306; and an example ultrasound image 1308.

The example ultrasound image 1302 depicts an anatomy of a second medical patient. A common carotid artery of the second medical patient was flagged by a user-provided click, and the spatial coordinate or intra-image location indicated by such user-provided click is conceptually represented by the red “X” superimposed on the example ultrasound image 1302. In like fashion, a thyroid of the second medical patient was flagged by a user-provided click, and the spatial coordinate or intra-image location indicated by such user-provided click is conceptually represented by the blue “X” superimposed on the example ultrasound image 1302.

The example ultrasound image 1304 depicts an anatomy of a third medical patient. A common carotid artery of the third medical patient was flagged by a user-provided click, and the spatial coordinate or intra-image location indicated by such user-provided click is conceptually represented by the red “X” superimposed on the example ultrasound image 1304. In like fashion, a thyroid of the third medical patient was flagged by a user-provided click, and the spatial coordinate or intra-image location indicated by such user-provided click is conceptually represented by the blue “X” superimposed on the example ultrasound image 1304.

The example ultrasound image 1306 depicts an anatomy of a fourth medical patient. A common carotid artery of the fourth medical patient was flagged by a user-provided click, and the spatial coordinate or intra-image location indicated by such user-provided click is conceptually represented by the red “X” superimposed on the example ultrasound image 1306. In like fashion, a thyroid of the fourth medical patient was flagged by a user-provided click, and the spatial coordinate or intra-image location indicated by such user-provided click is conceptually represented by the blue “X” superimposed on the example ultrasound image 1306.

The example ultrasound image 1308 depicts an anatomy of a fifth medical patient. A common carotid artery of the fifth medical patient was flagged by a user-provided click, and the spatial coordinate or intra-image location indicated by such user-provided click is conceptually represented by the red “X” superimposed on the example ultrasound image 1308. In like fashion, a thyroid of the fifth medical patient was flagged by a user-provided click, and the spatial coordinate or intra-image location indicated by such user-provided click is conceptually represented by the blue “X” superimposed on the example ultrasound image 1308.

As can be seen, because the example ultrasound images 1302, 1304, 1306, and 1308 correspond to different medical patients, they depict common carotid arteries and thyroids with varying physical characteristics (e.g., with different sizes, with different shapes or contours, with different shadings).

Now, various embodiments described herein were applied to the ultrasound image 1200 and to the example ultrasound images 1302, 1304, 1306, and 1308, so as to generate an ensembled heat map that localizes a thyroid within the ultrasound image 1200.

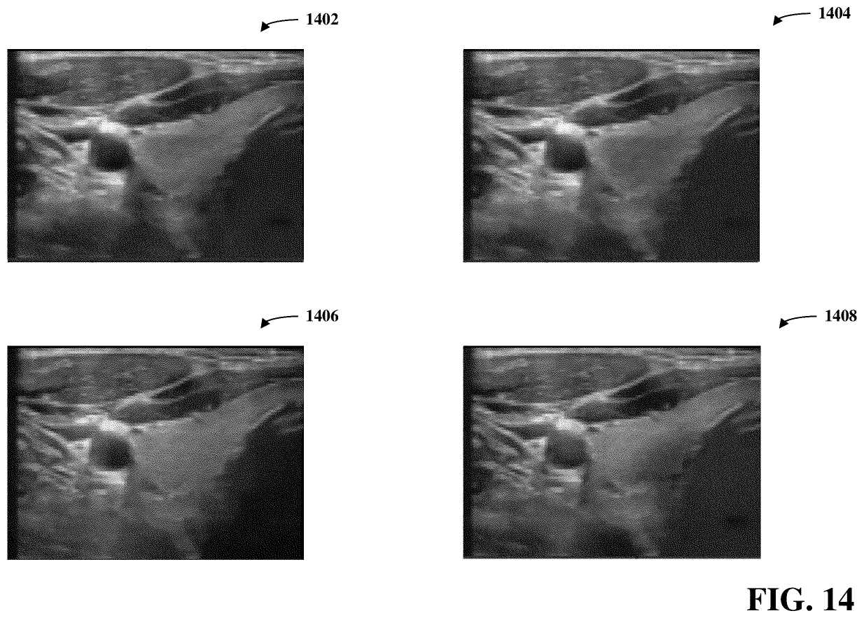

In particular, the ultrasound image 1200 was broken into patches, and each patch was passed through an embedder neural network. Moreover, whatever patch was flagged for the thyroid (e.g., indicated by the blue “X”) in the example ultrasound image 1302 was also passed through the embedder neural network. Accordingly, as shown in FIG. 14, a heat map 1402 was generated, by computing similarity scores between the embedding of that flagged patch and each of the embeddings of the patches of the ultrasound image 1200. Redder colors in the heat map 1402 indicate patches of the ultrasound image 1200 that are more similar to the flagged patch of the example ultrasound image 1302 that is known or deemed to depict the thyroid of the second medical patient. In contrast, bluer or blacker colors in the heat map 1402 indicate patches of the ultrasound image 1200 that are less similar to the flagged patch of the example ultrasound image 1302 that is known or deemed to depict the thyroid of the second medical patient.

This procedure was repeated for the example ultrasound image 1304, thereby yielding a heat map 1404. That is, whatever patch was flagged for the thyroid (e.g., indicated by the blue “X”) in the example ultrasound image 1304 was passed through the embedder neural network, and the heat map 1404 was generated, by computing similarity scores between the embedding of that flagged patch and each of the embeddings of the patches of the ultrasound image 1200. Redder colors in the heat map 1404 indicate patches of the ultrasound image 1200 that are more similar to the flagged patch of the example ultrasound image 1304 that is known or deemed to depict the thyroid of the third medical patient. In contrast, bluer or blacker colors in the heat map 1404 indicate patches of the ultrasound image 1200 that are less similar to the flagged patch of the example ultrasound image 1304 that is known or deemed to depict the thyroid of the third medical patient.

This procedure was also repeated for the example ultrasound image 1306, thereby yielding a heat map 1406. That is, whatever patch was flagged for the thyroid (e.g., indicated by the blue “X”) in the example ultrasound image 1306 was passed through the embedder neural network, and the heat map 1406 was generated, by computing similarity scores between the embedding of that flagged patch and each of the embeddings of the patches of the ultrasound image 1200. Redder colors in the heat map 1406 indicate patches of the ultrasound image 1200 that are more similar to the flagged patch of the example ultrasound image 1306 that is known or deemed to depict the thyroid of the fourth medical patient. In contrast, bluer or blacker colors in the heat map 1406 indicate patches of the ultrasound image 1200 that are less similar to the flagged patch of the example ultrasound image 1306 that is known or deemed to depict the thyroid of the fourth medical patient.

This procedure was again repeated for the example ultrasound image 1308, thereby yielding a heat map 1408. That is, whatever patch was flagged for the thyroid (e.g., indicated by the blue “X”) in the example ultrasound image 1308 was passed through the embedder neural network, and the heat map 1408 was generated, by computing similarity scores between the embedding of that flagged patch and each of the embeddings of the patches of the ultrasound image 1200. Redder colors in the heat map 1408 indicate patches of the ultrasound image 1200 that are more similar to the flagged patch of the example ultrasound image 1308 that is known or deemed to depict the thyroid of the fifth medical patient. In contrast, bluer or blacker colors in the heat map 1408 indicate patches of the ultrasound image 1200 that are less similar to the flagged patch of the example ultrasound image 1308 that is known or deemed to depict the thyroid of the fifth medical patient.

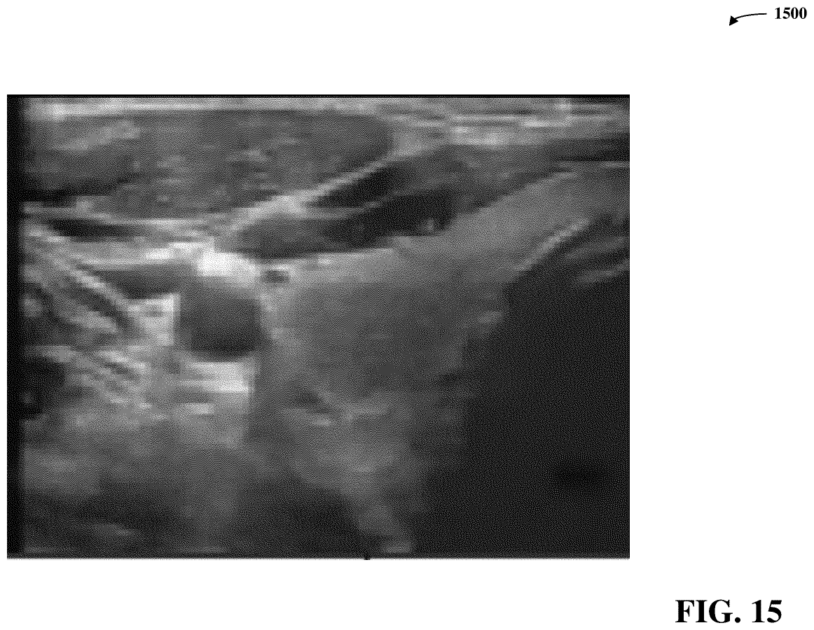

FIG. 15 shows an ensembled heat map 1500. The ensembled heat map 1500 was computed by averaging the heat maps 1402, 1404, 1406, and 1408. Accordingly, the ensembled heat map 1500 can be considered as indicating which patches of the ultrasound image 1200 are more (e.g., indicated by redder colors) or less (e.g., indicated by bluer or blacker colors) likely to depict a thyroid. Note that the heat map 1406 is very unlike the heat maps 1402, 1404, and 1408. In other words, the heat map 1406 can be considered as outlying or unreliable. This can be due to an unexpected visual characteristic of the example ultrasound image 1306. Indeed, the user-provided click for the thyroid in the example ultrasound image 1306 indicated or flagged an unusually dark location for a thyroid. Such usual darkness caused the heat map 1406 to be outlying or unreliable. However, note how the ensembled heat map 1500 strongly identifies logical locations for the thyroid of the first medical patient, despite the outlying nature or unreliability of the heat map 1406. In other words, the outlying nature or unreliability of the heat map 1406 was averaged out by aggregating all of the heat maps 1402, 1404, 1406, and 1408 together.

Now, various embodiments described herein were also applied to the ultrasound image 1200 and to the example ultrasound images 1302, 1304, 1306, and 1308, so as to generate an ensembled heat map that localizes a common carotid artery within the ultrasound image 1200.

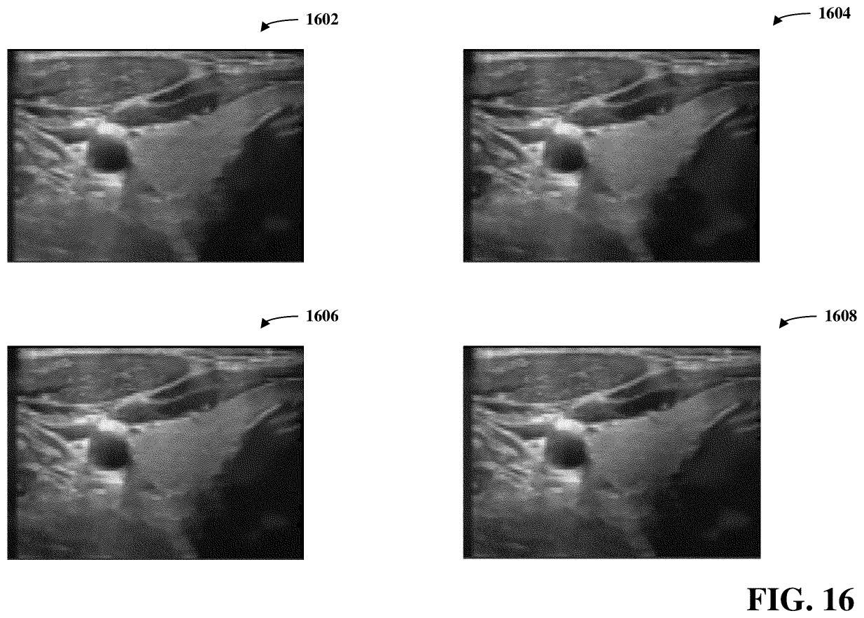

In particular, whatever patch was flagged for the common carotid artery (e.g., indicated by the red “X”) in the example ultrasound image 1302 was passed through the embedder neural network, and, as shown in FIG. 16, a heat map 1602 was generated, by computing similarity scores between the embedding of that flagged patch and each of the embeddings of the patches of the ultrasound image 1200. Redder colors in the heat map 1602 indicate patches of the ultrasound image 1200 that are more similar to the flagged patch of the example ultrasound image 1302 that is known or deemed to depict the common carotid artery of the second medical patient. In contrast, bluer or blacker colors in the heat map 1602 indicate patches of the ultrasound image 1200 that are less similar to the flagged patch of the example ultrasound image 1302 that is known or deemed to depict the common carotid artery of the second medical patient.

This procedure was repeated for the example ultrasound image 1304, thereby yielding a heat map 1604. That is, whatever patch was flagged for the common carotid artery (e.g., indicated by the red “X”) in the example ultrasound image 1304 was passed through the embedder neural network, and the heat map 1604 was generated, by computing similarity scores between the embedding of that flagged patch and each of the embeddings of the patches of the ultrasound image 1200. Redder colors in the heat map 1604 indicate patches of the ultrasound image 1200 that are more similar to the flagged patch of the example ultrasound image 1304 that is known or deemed to depict the common carotid artery of the third medical patient. In contrast, bluer or blacker colors in the heat map 1604 indicate patches of the ultrasound image 1200 that are less similar to the flagged patch of the example ultrasound image 1304 that is known or deemed to depict the common carotid artery of the third medical patient.

This procedure was also repeated for the example ultrasound image 1306, thereby yielding a heat map 1606. That is, whatever patch was flagged for the common carotid artery (e.g., indicated by the red “X”) in the example ultrasound image 1306 was passed through the embedder neural network, and the heat map 1606 was generated, by computing similarity scores between the embedding of that flagged patch and each of the embeddings of the patches of the ultrasound image 1200. Redder colors in the heat map 1606 indicate patches of the ultrasound image 1200 that are more similar to the flagged patch of the example ultrasound image 1306 that is known or deemed to depict the common carotid artery of the fourth medical patient. In contrast, bluer or blacker colors in the heat map 1606 indicate patches of the ultrasound image 1200 that are less similar to the flagged patch of the example ultrasound image 1306 that is known or deemed to depict the common carotid artery of the fourth medical patient.

This procedure was again repeated for the example ultrasound image 1308, thereby yielding a heat map 1608. That is, whatever patch was flagged for the common carotid artery (e.g., indicated by the red “X”) in the example ultrasound image 1308 was passed through the embedder neural network, and the heat map 1608 was generated, by computing similarity scores between the embedding of that flagged patch and each of the embeddings of the patches of the ultrasound image 1200. Redder colors in the heat map 1608 indicate patches of the ultrasound image 1200 that are more similar to the flagged patch of the example ultrasound image 1308 that is known or deemed to depict the common carotid artery of the fifth medical patient. In contrast, bluer or blacker colors in the heat map 1608 indicate patches of the ultrasound image 1200 that are less similar to the flagged patch of the example ultrasound image 1308 that is known or deemed to depict the common carotid artery of the fifth medical patient.

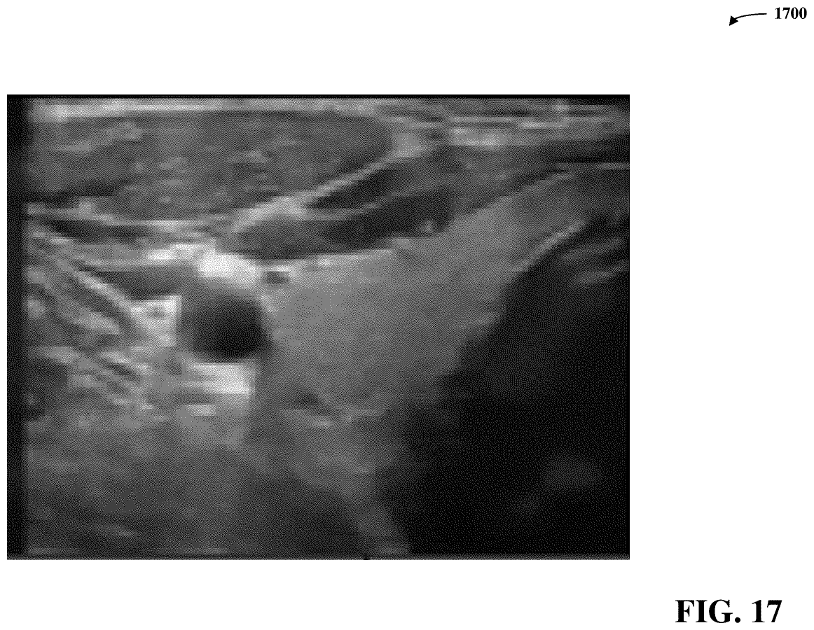

FIG. 17 shows an ensembled heat map 1700. The ensembled heat map 1700 was computed by averaging the heat maps 1602, 1604, 1606, and 1608. Accordingly, the ensembled heat map 1700 can be considered as indicating which patches of the ultrasound image 1200 are more (e.g., indicated by redder colors) or less (e.g., indicated by bluer or blacker colors) likely to depict a common carotid artery.

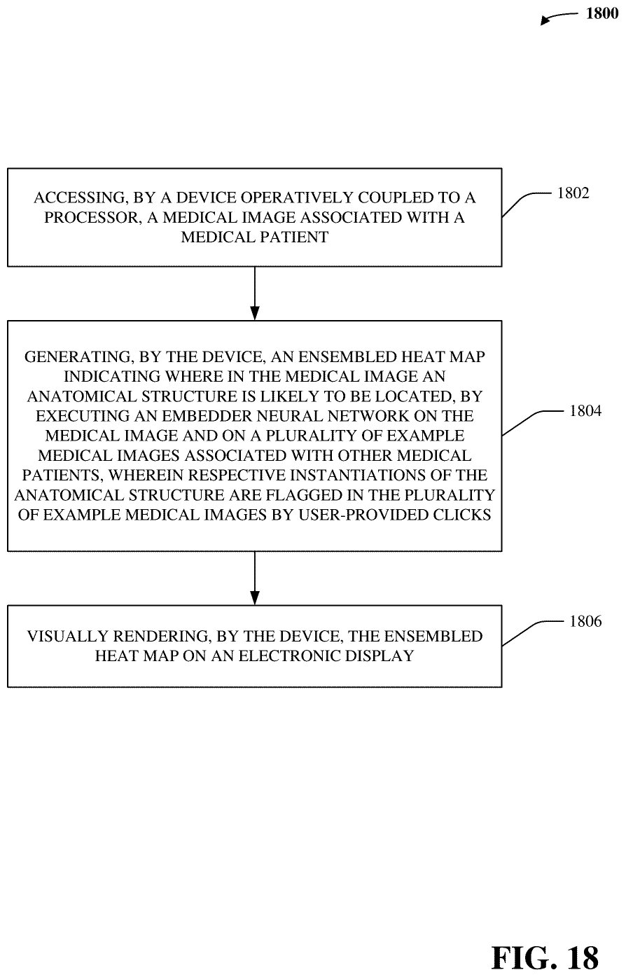

FIG. 18 illustrates a flow diagram of an example, non-limiting computer-implemented method 1800 that can facilitate ensembled querying of example images via deep learning embeddings in accordance with one or more embodiments described herein. In various cases, the ensembled visual querying system 102 can facilitate the computer-implemented method 1800.

In various embodiments, act 1802 can include accessing, by a device (e.g., via 112) operatively coupled to a processor (e.g., 108), a medical image (e.g., 104) associated with a medical patient.

In various aspects, act 1804 can include generating, by the device (e.g., via 116), an ensembled heat map (e.g., 404) indicating where in the medical image an anatomical structure (e.g., 106) is likely to be located, by executing an embedder neural network (e.g., 402) on the medical image and on a plurality of example medical images (e.g., 202) associated with other medical patients. In various cases, respective instantiations of the anatomical structure can be flagged in the plurality of example medical images by user-provided clicks (e.g., 204).

In various instances, act 1806 can include visually rendering, by the device (e.g., via 118), the ensembled heat map on an electronic display.

Although not explicitly shown in FIG. 18, the embedder neural network can be executed on the medical image and on the plurality of example medical images in patch-wise fashion. In particular, the computer-implemented method 1800 can further comprise: dividing, by the device (e.g., via 116), the medical image into a set of pixel or voxel patches (e.g., 502); and executing, by the device (e.g., via 116), the embedder neural network on each of the set of pixel or voxel patches, thereby yielding a set of patch embeddings (e.g., 504). Furthermore, the computer-implemented method 1800 can further comprise, for each example medical image (e.g., 202(n)) in the plurality of example medical images: identifying, by the device (e.g., via 116), within the example medical image, and based on a user-provided click (e.g., 204(n)) of the example medical image, a flagged pixel or voxel patch (e.g., 602(n)) that depicts an instantiation of the anatomical structure; executing, by the device (e.g., via 116), the embedder neural network on the flagged pixel or voxel patch, thereby yielding a flagged patch embedding (e.g., 604(n)); and computing, by the device (e.g., via 116), a heat map (e.g., 702(n)), based on similarity scores computed between the flagged patch embedding and each of the set of patch embeddings. In various cases, this can yield a plurality of heat maps (e.g., 702) respectively corresponding to the plurality of example medical images.

Although not explicitly shown in FIG. 18, the generating the ensembled heat map can be based on aggregating, by the device (e.g., via 116), the plurality of heat maps together.

Although various embodiments described herein have been discussed with reference to localization of anatomical structures depicted in medical images, this is a mere non-limiting example for case of explanation and illustration. In various aspects, various embodiments can be implemented to localize any suitable objects of interest (e.g., even objects that are not anatomical structures) depicted in any suitable images (e.g., even in non-medical images).

Various embodiments described herein can include a computer program product for facilitating ensembled querying of example images via deep learning embeddings. In various aspects, the computer program product can comprise a non-transitory computer-readable memory (e.g., 110) having program instructions embodied therewith. In various instances, program instructions can be executable by a processor (e.g., 108) to cause the processor to: access an image (e.g., 104); and localize an object of interest (e.g., 106) depicted in the image, by executing an embedder neural network (e.g., 402) on the image and on a plurality of example images (e.g., 202), wherein respective instantiations of the object of interest can be flagged in the plurality of example images by user-provided clicks (e.g., 204).

In various aspects, the instructions can be further executable to cause the processor to: divide the image into a set of pixel or voxel patches (e.g., 502); and execute the embedder neural network on each of the set of pixel or voxel patches, thereby yielding a set of patch embeddings (e.g., 504).

In various instances, the instructions can be further executable to cause the processor to, for each example image (e.g., 202(n)) in the plurality of example images: identify, within the example image and based on a user-provided click (e.g., 204(n)) of the example image, a flagged pixel or voxel patch (e.g., 602(n)) that depicts an instantiation of the object of interest; execute the embedder neural network on the flagged pixel or voxel patch, thereby yielding a flagged patch embedding (e.g., 604(n)); compute a heat map (e.g., 702(n)), based on similarity scores computed between the flagged patch embedding and each of the set of patch embeddings, thereby yielding a plurality of heat maps (e.g., 702) respectively corresponding to the plurality of example images; and aggregate the plurality of heat maps together, thereby yielding an ensembled heat map (e.g., 404).

In various cases, the instructions can be further executable to cause the processor to: visually render the ensembled heat map on an electronic display.

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ artificial intelligence to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g., support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z=(z₁, z₂, z₃, z₄, zₙ), to a confidence that the input belongs to a class, as by f(z)=confidence (class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

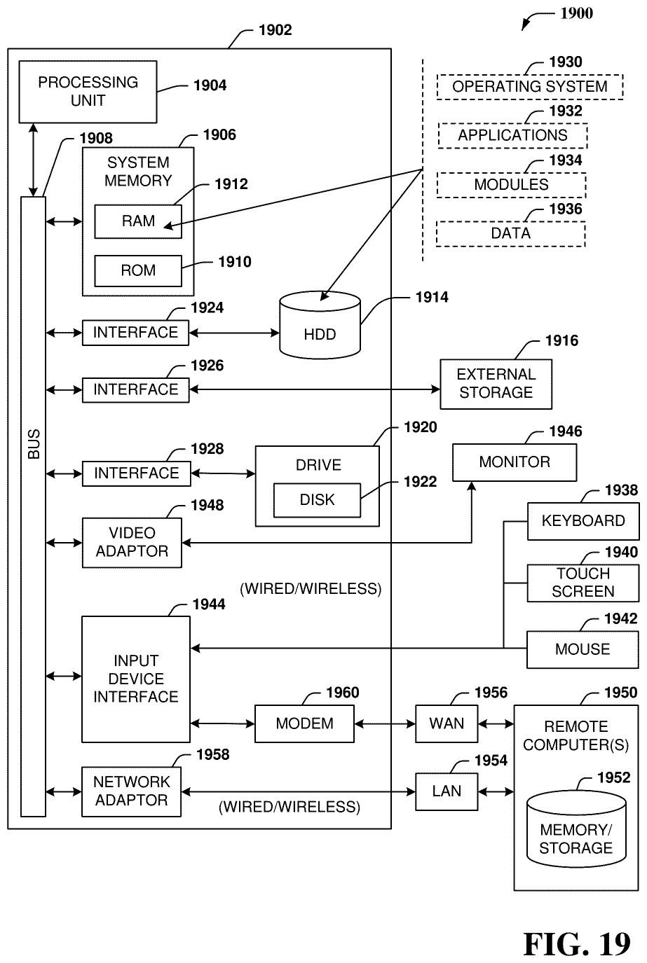

In order to provide additional context for various embodiments described herein, FIG. 19 and the following discussion are intended to provide a brief, general description of a suitable computing environment 1900 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per sc.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 19, the example environment 1900 for implementing various embodiments of the aspects described herein includes a computer 1902, the computer 1902 including a processing unit 1904, a system memory 1906 and a system bus 1908. The system bus 1908 couples system components including, but not limited to, the system memory 1906 to the processing unit 1904. The processing unit 1904 can be any of various commercially available processors. Dual microprocessors and other multi-processor architectures can also be employed as the processing unit 1904.

The system bus 1908 can be any of several types of bus structure that can further interconnect to a memory bus (with or without a memory controller), a peripheral bus, and a local bus using any of a variety of commercially available bus architectures. The system memory 1906 includes ROM 1910 and RAM 1912. A basic input/output system (BIOS) can be stored in a non-volatile memory such as ROM, erasable programmable read only memory (EPROM), EEPROM, which BIOS contains the basic routines that help to transfer information between elements within the computer 1902, such as during startup. The RAM 1912 can also include a high-speed RAM such as static RAM for caching data.

The computer 1902 further includes an internal hard disk drive (HDD) 1914 (e.g., EIDE, SATA), one or more external storage devices 1916 (e.g., a magnetic floppy disk drive (FDD) 1916, a memory stick or flash drive reader, a memory card reader, etc.) and a drive 1920, e.g., such as a solid state drive, an optical disk drive, which can read or write from a disk 1922, such as a CD-ROM disc, a DVD, a BD, etc. Alternatively, where a solid state drive is involved, disk 1922 would not be included, unless separate. While the internal HDD 1914 is illustrated as located within the computer 1902, the internal HDD 1914 can also be configured for external use in a suitable chassis (not shown). Additionally, while not shown in environment 1900, a solid state drive (SSD) could be used in addition to, or in place of, an HDD 1914. The HDD 1914, external storage device(s) 1916 and drive 1920 can be connected to the system bus 1908 by an HDD interface 1924, an external storage interface 1926 and a drive interface 1928, respectively. The interface 1924 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1902, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1912, including an operating system 1930, one or more application programs 1932, other program modules 1934 and program data 1936. All or portions of the operating system, applications, modules, or data can also be cached in the RAM 1912. The systems and methods described herein can be implemented utilizing various commercially available operating systems or combinations of operating systems.

Computer 1902 can optionally comprise emulation technologies. For example, a hypervisor (not shown) or other intermediary can emulate a hardware environment for operating system 1930, and the emulated hardware can optionally be different from the hardware illustrated in FIG. 19. In such an embodiment, operating system 1930 can comprise one virtual machine (VM) of multiple VMs hosted at computer 1902. Furthermore, operating system 1930 can provide runtime environments, such as the Java runtime environment or the .NET framework, for applications 1932. Runtime environments are consistent execution environments that allow applications 1932 to run on any operating system that includes the runtime environment. Similarly, operating system 1930 can support containers, and applications 1932 can be in the form of containers, which are lightweight, standalone, executable packages of software that include, e.g., code, runtime, system tools, system libraries and settings for an application.

Further, computer 1902 can be enable with a security module, such as a trusted processing module (TPM). For instance with a TPM, boot components hash next in time boot components, and wait for a match of results to secured values, before loading a next boot component. This process can take place at any layer in the code execution stack of computer 1902, e.g., applied at the application execution level or at the operating system (OS) kernel level, thereby enabling security at any level of code execution.

A user can enter commands and information into the computer 1902 through one or more wired/wireless input devices, e.g., a keyboard 1938, a touch screen 1940, and a pointing device, such as a mouse 1942. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1904 through an input device interface 1944 that can be coupled to the system bus 1908, but can be connected by other interfaces, such as a parallel port, an IEEE 1394 serial port, a game port, a USB port, an IR interface, a BLUETOOTH® interface, etc.

A monitor 1946 or other type of display device can be also connected to the system bus 1908 via an interface, such as a video adapter 1948. In addition to the monitor 1946, a computer typically includes other peripheral output devices (not shown), such as speakers, printers, etc.

The computer 1902 can operate in a networked environment using logical connections via wired or wireless communications to one or more remote computers, such as a remote computer(s) 1950. The remote computer(s) 1950 can be a workstation, a server computer, a router, a personal computer, portable computer, microprocessor-based entertainment appliance, a peer device or other common network node, and typically includes many or all of the elements described relative to the computer 1902, although, for purposes of brevity, only a memory/storage device 1952 is illustrated. The logical connections depicted include wired/wireless connectivity to a local area network (LAN) 1954 or larger networks, e.g., a wide area network (WAN) 1956. Such LAN and WAN networking environments are commonplace in offices and companies, and facilitate enterprise-wide computer networks, such as intranets, all of which can connect to a global communications network, e.g., the Internet.

When used in a LAN networking environment, the computer 1902 can be connected to the local network 1954 through a wired or wireless communication network interface or adapter 1958. The adapter 1958 can facilitate wired or wireless communication to the LAN 1954, which can also include a wireless access point (AP) disposed thereon for communicating with the adapter 1958 in a wireless mode.

When used in a WAN networking environment, the computer 1902 can include a modem 1960 or can be connected to a communications server on the WAN 1956 via other means for establishing communications over the WAN 1956, such as by way of the Internet. The modem 1960, which can be internal or external and a wired or wireless device, can be connected to the system bus 1908 via the input device interface 1944. In a networked environment, program modules depicted relative to the computer 1902 or portions thereof, can be stored in the remote memory/storage device 1952. It will be appreciated that the network connections shown are example and other means of establishing a communications link between the computers can be used.

When used in either a LAN or WAN networking environment, the computer 1902 can access cloud storage systems or other network-based storage systems in addition to, or in place of, external storage devices 1916 as described above, such as but not limited to a network virtual machine providing one or more aspects of storage or processing of information. Generally, a connection between the computer 1902 and a cloud storage system can be established over a LAN 1954 or WAN 1956 e.g., by the adapter 1958 or modem 1960, respectively. Upon connecting the computer 1902 to an associated cloud storage system, the external storage interface 1926 can, with the aid of the adapter 1958 or modem 1960, manage storage provided by the cloud storage system as it would other types of external storage. For instance, the external storage interface 1926 can be configured to provide access to cloud storage sources as if those sources were physically connected to the computer 1902.

The computer 1902 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

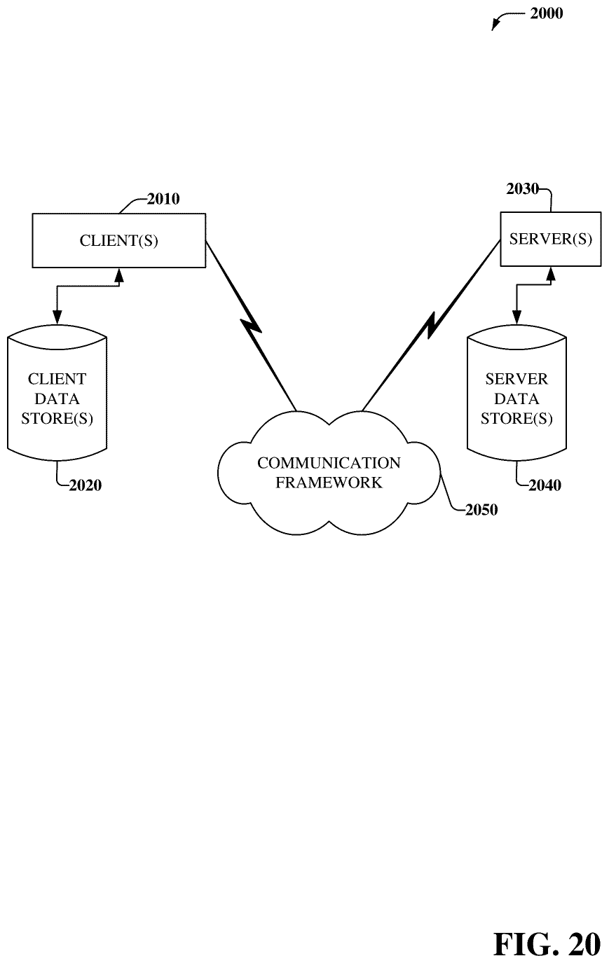

FIG. 20 is a schematic block diagram of a sample computing environment 2000 with which the disclosed subject matter can interact. The sample computing environment 2000 includes one or more client(s) 2010. The client(s) 2010 can be hardware or software (e.g., threads, processes, computing devices). The sample computing environment 2000 also includes one or more server(s) 2030. The server(s) 2030 can also be hardware or software (e.g., threads, processes, computing devices). The servers 2030 can house threads to perform transformations by employing one or more embodiments as described herein, for example. One possible communication between a client 2010 and a server 2030 can be in the form of a data packet adapted to be transmitted between two or more computer processes. The sample computing environment 2000 includes a communication framework 2050 that can be employed to facilitate communications between the client(s) 2010 and the server(s) 2030. The client(s) 2010 are operably connected to one or more client data store(s) 2020 that can be employed to store information local to the client(s) 2010. Similarly, the server(s) 2030 are operably connected to one or more server data store(s) 2040 that can be employed to store information local to the servers 2030.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For case of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes computer-executable components stored in a non-transitory computer-readable memory, wherein the computer-executable components comprise:
an access component that accesses a medical image associated with a medical patient; and
a localization component that generates an ensembled heat map indicating where in the medical image an anatomical structure is likely to be located, by executing an embedder neural network on the medical image and on a plurality of example medical images associated with other medical patients, wherein respective instantiations of the anatomical structure are flagged in the plurality of example medical images by user-provided clicks.

2. The system of claim 1, wherein the embedder neural network is executed on the medical image and on the plurality of example medical images in patch-wise fashion.

3. The system of claim 2, wherein the localization component:
divides the medical image into a set of pixel or voxel patches; and
executes the embedder neural network on each of the set of pixel or voxel patches, thereby yielding a set of patch embeddings.

4. The system of claim 3, wherein, for each example medical image in the plurality of example medical images, the localization component:
identifies, within the example medical image and based on a user-provided click of the example medical image, a flagged pixel or voxel patch that depicts an instantiation of the anatomical structure;
executes the embedder neural network on the flagged pixel or voxel patch, thereby yielding a flagged patch embedding; and
computes a heat map, based on similarity scores computed between the flagged patch embedding and each of the set of patch embeddings, thereby yielding a plurality of heat maps respectively corresponding to the plurality of example medical images.

5. The system of claim 4, wherein the similarity scores are cosine similarities.

6. The system of claim 4, wherein the localization component generates the ensembled heat map by aggregating the plurality of heat maps together.

7. The system of claim 1, wherein the computer-executable components further comprise:
a display component that visually renders the ensembled heat map on an electronic display.

8. The system of claim 1, wherein the embedder neural network is trained in unsupervised fashion in an encoder-decoder deep learning pipeline or via a self-distillation-no-labels technique, or wherein the embedder neural network is an encoder of a pre-trained vision transformer.

9. A computer-implemented method, comprising:
accessing, by a device operatively coupled to a processor, a medical image associated with a medical patient; and
generating, by the device, an ensembled heat map indicating where in the medical image an anatomical structure is likely to be located, by executing an embedder neural network on the medical image and on a plurality of example medical images associated with other medical patients, wherein respective instantiations of the anatomical structure are flagged in the plurality of example medical images by user-provided clicks.

10. The computer-implemented method of claim 9, wherein the embedder neural network is executed on the medical image and on the plurality of example medical images in patch-wise fashion.

11. The computer-implemented method of claim 10, further comprising:
dividing, by the device, the medical image into a set of pixel or voxel patches; and
executing, by the device, the embedder neural network on each of the set of pixel or voxel patches, thereby yielding a set of patch embeddings.

12. The computer-implemented method of claim 11, further comprising, for each example medical image in the plurality of example medical images:
identifying, by the device, within the example medical image, and based on a user-provided click of the example medical image, a flagged pixel or voxel patch that depicts an instantiation of the anatomical structure;
executing, by the device, the embedder neural network on the flagged pixel or voxel patch, thereby yielding a flagged patch embedding; and
computing, by the device, a heat map, based on similarity scores computed between the flagged patch embedding and each of the set of patch embeddings, thereby yielding a plurality of heat maps respectively corresponding to the plurality of example medical images.

13. The computer-implemented method of claim 12, wherein the similarity scores are cosine similarities.

14. The computer-implemented method of claim 12, wherein the generating the ensembled heat map is based on aggregating, by the device, the plurality of heat maps together.

15. The computer-implemented method of claim 9, further comprising:
visually rendering, by the device, the ensembled heat map on an electronic display.

16. The computer-implemented method of claim 9, wherein the embedder neural network is trained in unsupervised fashion in an encoder-decoder deep learning pipeline or via a self-distillation-no-labels technique, or wherein the embedder neural network is an encoder of a pre-trained vision transformer.

17. A computer program product for facilitating ensembled querying of example images via deep learning embeddings, the computer program product comprising a non-transitory computer-readable memory having program instructions embodied therewith, the program instructions executable by a processor to cause the processor to:
access an image; and
localize an object of interest depicted in the image, by executing an embedder neural network on the image and on a plurality of example images, wherein respective instantiations of the object of interest are flagged in the plurality of example images by user-provided clicks.

18. The computer program product of claim 17, wherein the program instructions are further executable to cause the processor to:
divide the image into a set of pixel or voxel patches; and
execute the embedder neural network on each of the set of pixel or voxel patches, thereby yielding a set of patch embeddings.

19. The computer program product of claim 18, wherein the program instructions are further executable to cause the processor to, for each example image in the plurality of example images:
identify, within the example image and based on a user-provided click of the example image, a flagged pixel or voxel patch that depicts an instantiation of the object of interest;
execute the embedder neural network on the flagged pixel or voxel patch, thereby yielding a flagged patch embedding;
compute a heat map, based on similarity scores computed between the flagged patch embedding and each of the set of patch embeddings, thereby yielding a plurality of heat maps respectively corresponding to the plurality of example images; and
aggregate the plurality of heat maps together, thereby yielding an ensembled heat map.

20. The computer program product of claim 19, wherein the program instructions are further executable to cause the processor to:
visually render the ensembled heat map on an electronic display.
